(19) United States
(12) Patent Application Publication
(10) Pub. No.: US 2015/0315130 A1
(43) Pub. Date: Nov. 5, 2015

(54) New positive allosteric modulators of nicotinic acetylcholine receptor

(71) Applicant: H. LUNDBECK A/S, Valby (DK)

(72) Inventors: Jørgen Eskildsen, København S (DK); Anette Graven Sams, Værløse (DK); Ask Püschl, Frederiksberg C. (DK)

(73) Assignee: H. Lundbeck A/S, Valby (DK)

(21) Appl. No.: 14/650,633

(22) PCT Filed: Dec. 9, 2013
(86) PCT No.: PCT/EP2013/075925

§ 371 Date: Jun. 9, 2015

Related U.S. Application Data
(60) Provisional application No. 61/735,077, filed on Dec. 10, 2012.

(30) Foreign Application Priority Data
Dec. 10, 2012 (DK) PA 2012 00778
Jun. 12, 2013 (DK) PA 2013 00355

(51) Int. Cl. C07C 233/60; C07C 233/59

(52) U.S. Cl. CPC C07C 233/60; C07C 233/59; C07C 2101/02

(57) ABSTRACT

The present invention relates to compounds useful in therapy, to compositions comprising said compounds, and to methods of treating diseases comprising administration of said compounds. The compounds referred to are positive allosteric modulators (PAMs) of the nicotinic acetylcholine α7 receptor.

### FIELD OF THE INVENTION

The present invention relates to compounds useful in therapy, to compositions comprising said compounds, and to methods of treating diseases comprising administration of said compounds. The compounds referred to are positive allosteric modulators (PAMs) of the nicotinic acetylcholine α7 receptor.

### BACKGROUND OF THE INVENTION

Nicotinic acetylcholine receptors (nAChRs) belong to the super family of ligand gated ionic channels, and gate the flow of cations including calcium. The nAChRs are endogenously activated by acetylcholine (ACh) and can be divided into nicotinic receptors of the neuromuscular junction and neuronal nicotinic receptors (NNRs). The NNRs are widely expressed throughout the central nervous system (CNS) and the peripheral nervous system (PNS). The NNRs have been suggested to play an important role in CNS function by modulating the release of many neurotransmitters, for example, ACh, norepinephrine, dopamine, serotonin, and GABA, among others, resulting in a wide range of physiological effects.

Seventeen subunits of nAChRs have been reported to date, which are identified as α2-α10, β1-β4, γ, δ and ε. From these subunits, nine subunits, α2 through α7 and β2 through β4, prominently exist in the mammalian brain. Many functionally distinct nAChR complexes exist, for example five α7 subunits can form a receptor as a homomeric functional pentamer or combinations of different subunits can form heteromeric receptors such as α4β2 and α3β4 receptors (Gotti, C. et al., Prog. Neurobiol., 2004, 74: 363-396; Gotti, C. et al., Biochemical Pharmacology, 2009, 78: 703-711)

The homomeric α7 receptor is one of the most abundant NNRs, along with α4β2 receptors, in the brain, wherein it is heavily expressed in the hippocampus, cortex, thalamic nuclei, ventral tegmental area and substantia nigra (Broad, L. M. et al., Drugs of the Future, 2007, 32(2): 161-170, Poorthuis R B, Biochem Pharmacol. 2009, 1; 78(7):668-76).

The role of α7 NNR in neuronal signalling has been actively investigated. The α7 NNRs have been demonstrated to regulate interneuron excitability and modulate the release of excitatory as well as inhibitory neurotransmitters. In addition, α7 NNRs have been reported to be involved in neuroprotective effects in experimental models of cellular damage (Shimohama, S., Biol Pharm Bull. 2009, 32(3):332-6).

Studies have shown that α7 subunits, when expressed recombinant in-vitro, activate and desensitize rapidly, and exhibit relatively higher calcium permeability compared to other NNR combinations (Papke, R. L. et al., J Pharmacol Exp Ther. 2009, 329(2):791-807).

The NNRs, in general, are involved in various cognitive functions, such as learning, memory and attention, and therefore in CNS disorders, e.g. Alzheimer's disease (AD), Parkinson's disease (PD), attention deficit hyperactivity disorder (ADHD), Tourette's syndrome, schizophrenia, bipolar disorder, pain and tobacco dependence (Keller, J. J. et al., Behav. Brain Res. 2005, 162: 143-52; Haydar, S. N. et al., Curr Top Med Chem. 2010; 10(2):144-52).

The α7 NNRs in particular, have also been linked to cognitive disorders including, for example, ADHD, autism spectrum disorders, AD, mild cognitive impairment (MCI), age associated memory impairment (AAMI) senile dementia, frontotemporal lobar degeneration, HIV associated dementia (HAD), HIV associated cognitive impairment (HIV-CI), Pick's disease, dementia associated with Lewy bodies, cognitive impairment associated with Multiple Sclerosis, Vascular Dementia, cognitive impairment in epilepsy, cognitive impairment associated with fragile X, cognitive impairment associated with Friedreich's Ataxia, and dementia associated with Down's syndrome, as well as cognitive impairment associated with schizophrenia. In addition, α7-NNRs have been shown to be involved in the neuroprotective effects of nicotine both in vitro (Jonnala, R. B. et al., J. Neurosci. Res., 2001, 66: 565-572) and in vivo (Shimohama, S., Brain Res., 1998, 779: 359-363) as well as in pain signalling. More particularly, neurodegeneration underlies several progressive CNS disorders, including, but not limited to, AD, PD, amyotrophic lateral sclerosis, Huntington's disease, dementia with Lewy bodies, as well as diminished CNS function resulting from traumatic brain injury. For example, the impaired function of α7 NNRs by beta-amyloid peptides linked to AD has been implicated as a key factor in development of the cognitive deficits associated with the disease (Liu, Q.-S., et al., PNAS, 2001, 98: 4734-4739). Thus, modulating the activity of α7 NNRs demonstrates promising potential to prevent or treat a variety of diseases indicated above, such as AD, other dementias, other neurodegenerative diseases, schizophrenia and neurodegeneration, with an underlying pathology that involves cognitive function including, for example, aspects of learning, memory, and attention (Thomsen, M. S. et al., Curr Pharm Des. 2010 January; 16(3):323-43; Olincy, A. et al., Arch Gen Psychiatry. 2006, 63(6):630-8; Deutsch, S.I., Clin Neuropharmacol. 2010, 33(3):114-20; Feuerbach, D., Neuropharmacology. 2009, 56(1): 254-63).

The NNR ligands, including α7 ligands, have also been implicated in weight control, diabetis inflammation, obsessive-compulsive disorder (OCD), angiogenesis and as potential analgesics (Marrero, M. B. et al., J. Pharmacol. Exp. Ther. 2010, 332(1):173-80; Vincler, M., Exp. Opin. Invest. Drugs, 2005, 14 (10): 1191-1198; Rosas-Ballina, M., J. Intern Med. 2009 265(6):663-79; Arias, H. R., Int. J. Biochem. Cell Biol. 2009, 41(7):1441-51; Tizabi, Y., Biol Psychiatry. 2002, 51(2):164-71).

Nicotine is known to enhance attention and cognitive performance, reduced anxiety, enhanced sensory gating, and analgesia and neuroprotective effects when administered. Such effects are mediated by the non-selective effect of nicotine at multiple nicotinic receptor subtypes. However, nicotine also exerts adverse events, such as cardiovascular and gastrointestinal problems (Karaconji, I. B. et al., Arh Hig Rada Toksikol. 2005, 56(4):363-71). Consequently, there is a need to identify subtype-selective compounds that retain the beneficial effects of nicotine, or an NNR ligand, while eliminating or decreasing adverse effects.

Examples of reported NNR ligands are α7 NNR agonists, such as DMXB-A, SSR180711 and ABT-107, which have shown some beneficial effects on cognitive processing both in rodents and humans (see for example Hajos, M. et al., J Pharmacol Exp Ther. 2005, 312: 1213-22; Olincy, A. et al., Arch Gen Psychiatry. 2006 63(6):630-8; Pichat, P., et al., Neuropsychopharmacology. 2007 32(1):17-34; Bitner, R. S., J Pharmacol Exp Ther. 2010 1; 334(3):875-86). In addition, modulation of α7 NNRs have been reported to improve negative symptoms in patients with schizophrenia (Freedman, R. et al., Am J Psychiatry. 2008 165(8):1040-7).

Despite the beneficial effects of NNR ligands, it remains uncertain whether chronic treatment with agonists affecting NNRs may provide suboptimal benefit due to sustained activation and desensitization of the NNRs, in particular the α7 NNR subtype. In contrast to agonists, administering a positive allosteric modulator (PAM) can reinforce endogenous cholinergic transmission without directly stimulating the target receptor. Nicotinic PAMs can selectively modulate the activity of ACh at NNRs, preserving the activation and deactivation kinetics of the receptor. Accordingly, α7 NNR-selective PAMs have emerged (Faghih, R., Recent Pat CNS Drug Discov. 2007, 2(2):99-106).

Consequently, it would be beneficial to increase α7 NNR function by enhancing the effect of the endogenous neurotransmitter acetylcholine via PAMs. This could reinforce the endogenous cholinergic neurotransmission without directly activating α7 NNRs, like agonists. Indeed, PAMs for enhancing channel activity have been proven clinically successful for GABAa receptors where benzodiazepines and barbiturates, behave as PAMs acting at distinct sites (Hevers, W. et al., Mol. Neurobiol. 1998, 18: 35-86).

To date, only a few NNR PAMs are known, such as 5-hydroxyindole (5-HI), ivermectin, galantamine, and SLURP-1, a peptide derived from acetylcholinesterase (AChE). Genistein, a kinase inhibitor was also reported to increase α7 responses. PNU-120596, a urea derivative, was reported to increase the potency ACh as well as improve auditory gating deficits induced by amphetamine in rats. Also, NS1738, JNJ-1930942 and compound 6 have been reported to potentiate the response of ACh and exert beneficial effect in experimental models of sensory and cognitive processing in rodents. Other NNR PAMs include derivatives of quinuclidine, indole, benzopyrazole, thiazole, and benzoisothiazoles (Hurst, R. S. et al., J. Neurosci. 2005, 25: 4396-4405; Faghih, R., Recent Pat CNS Drug Discov. 2007, 2(2):99-106; Timmermann, D. B., J. Pharmacol. Exp. Ther. 2007, 323(1):294-307; Ng, H. J. et al., Proc. Natl. Acad. Sci. USA. 2007, 8; 104(19):8059-64; Dinklo, T., J. Pharmacol. Exp. Ther. 2011, 336(2):560-74).

WO 2009/043784 recites compounds of the overall structure

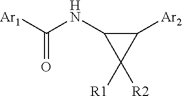

which compounds are said to be PAMs of the α7 NNR.

The α7 NNR PAMs presently known generally demonstrate weak activity, have a range of non-specific effects, or can only achieve limited access to the central nervous system where α7 NNRs are abundantly expressed. Accordingly, it would be beneficial to identify and provide new PAM compounds of α7 NNRs and compositions for treating diseases and disorders wherein α7 NNRs are involved. It would further be particularly beneficial if such compounds can provide improved efficacy of treatment while reducing adverse effects associated with compounds targeting neuronal nicotinic receptors by selectively modulating α7 NNRs.

The compounds (1S,2S)-2-Phenyl-cyclopropanecarboxylic acid{(R)-1-[4-(2-ethyl-butoxy)-2-methoxy-phenyl]-2-hydroxy-ethyl}-amide; (1S,2S)-2-Phenyl-cyclopropanecarboxylic acid ((R)-2-hydroxy-1-phenyl-ethyl)-amide and (1S,2S)-2-Phenyl-cyclopropanecarboxylic acid ((S)-1-phenyl-ethyl)-amide are disclosed in WO 2011/044195; Cho et al., J. Med. Chem. 2009, 52: 1885-1902; J. Am. Chem. Soc. 1991, 113: 8166-8167 and J. Am. Chem. Soc. 1991, 113: 726-728 respectively, and cited for activities that are not associated with modulation of the α7 NNR.

### SUMMARY OF THE INVENTION

The objective of the present invention is to provide compounds that are positive allosteric modulators (PAMs) of the nicotinic acetylcholine receptor subtype α7.

The compounds of the present invention are defined by formula [I] below:

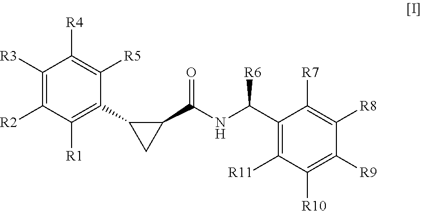

wherein R1, R2, R3, R4 and R5 are selected independently of each other from H and fluorine;
R6 is selected from methyl, methoxymethyl, hydroxymethyl and hydroxyethyl;
R7, R8, R9, R10 and R11 are selected independently of each other from H, C₁₋₆alkyl, C₂₋₆alkenyl, C₂₋₆alkynyl, C₁₋₆alkoxy, hydroxy, cyano, NR12R13, C₁₋₆alkylsulfonyl, halogen and OR14, wherein said C₁₋₆alkyl, C₂₋₆alkenyl, C₂₋₆alkynyl or C₁₋₆alkoxy is optionally substituted with one or more substituents selected from chlorine, fluorine, C₁₋₆alkoxy, cyano and NR12R13;
R12 and R13 independently represent hydrogen, C₁₋₆alkyl, C₂₋₆alkenyl and C₂₋₆alkynyl;
R14 represents a monocyclic saturated ring moiety having 4-6 ring atoms wherein one of said ring atoms is O and the other ring atoms are C;
and pharmaceutically acceptable salts thereof;
with the proviso that the compound of formula [I] is other than
(1S,2S)-2-Phenyl-cyclopropanecarboxylic acid{(R)-1-[4-(2-ethyl-butoxy)-2-methoxy-phenyl]-2-hydroxy-ethyl}-amide;
(1S,2S)-2-Phenyl-cyclopropanecarboxylic acid ((R)-2-hydroxy-1-phenyl-ethyl)-amide;
(1S,2S)-2-Phenyl-cyclopropanecarboxylic acid ((S)-1-phenyl-ethyl)-amide.

In one embodiment, the invention relates to a compound according to formula [I], and pharmaceutically acceptable salts thereof, for use as a medicament.

In one embodiment, the invention relates to a compound according to formula [I], and pharmaceutically acceptable salts thereof, for use in the treatment of a disease or disorder selected from psychosis; schizophrenia; cognitive disorders; cognitive impairment associated with schizophrenia; attention deficit hyperactivity disorder (ADHD); autism spectrum disorders, Alzheimer's disease (AD); mild cognitive impairment (MCI); age associated memory impairment (AAMI); senile dementia; AIDS dementia; Pick's disease; dementia associated with Lewy bodies; dementia associated with Down's syndrome; Huntington's disease; Parkinson's disease (PD); obsessive-compulsive disorder (OCD); traumatic brain injury; epilepsy; post-traumatic stress; Wernicke-Korsakoff syndrome (WKS); post-traumatic amnesia; cognitive deficits associated with depression; diabetes, weight control, inflammatory disorders, reduced angiogenesis; amyotrophic lateral sclerosis and pain.

In one embodiment, the invention relates to a pharmaceutical composition comprising a compound according to formula [I] and pharmaceutically acceptable salts thereof, and one or more pharmaceutically acceptable carrier or excipient.

In one embodiment, the invention relates to a kit comprising a compound according to formula [I], and pharmaceutically acceptable salts thereof, together with a compound selected from the list consisting of acetylcholinesterase inhibitors; glutamate receptor antagonists; dopamine transport inhibitors; noradrenalin transport inhibitors; D2 antagonists; D2 partial agonists; PDE10 antagonists; 5-HT2A antagonists; 5-HT6 antagonists; KCNQ antagonists; lithium; sodium channel blockers and GABA signaling enhancers.

### DEFINITIONS

In the present context, “optionally substituted” means that the indicated moiety may or may not be substituted, and when substituted is mono-, di-, or tri-substituted, such as with 1, 2 or 3 substituents. In some instances, the substituent is independently selected from the group consisting of C₁₋₆alkyl, C₂₋₆alkenyl, C₂₋₆alkynyl, C₁₋₆alkoxy, hydroxy and halogen. It is understood that where no substituents are indicated for an “optionally substituted” moiety, then the position is held by a hydrogen atom.

In the present context, “alkyl” is intended to indicate a straight, branched and/or cyclic saturated hydrocarbon. In particular “C₁₋₆alkyl” is intended to indicate such hydrocarbon having 1, 2, 3, 4, 5 or 6 carbon atoms. Examples of C₁₋₆alkyl include methyl, ethyl, propyl, butyl, pentyl, hexyl, cyclopropyl, cyclobutyl, cyclopentyl, cyclohexyl, methylcyclopropyl, 2-methylpropyl and tert-butyl. Examples of substituted C₁₋₆alkyl include e.g. fluoromethyl and hydroxymethyl.

In the present context, “alkenyl” is intended to indicate a non-aromatic, straight, branched and/or cyclic hydrocarbon comprising at least one carbon-carbon double bond. In particular “C₂₋₆alkenyl” is intended to indicate such hydrocarbon having 2, 3, 4, 5 or 6 carbon atoms. Examples of C₂₋₆alkenyl include ethenyl, 1-propenyl, 2-propenyl, 1-butenyl, 2-butenyl and 3-butenyl and cyclohexenyl.

In the present context, “alkynyl” is intended to indicate a non-aromatic, straight, branched and/or cyclic hydrocarbon comprising at least one carbon-carbon triple bond and optionally also one or more carbon-carbon double bonds. In particular “C₂₋₆alkynyl” is intended to indicate such hydrocarbon having 2, 3, 4, 5 or 6 carbon atoms. Examples of C₂₋₆alkynyl include ethynyl, 1-propynyl, 2-propynyl, 1-butynyl, 2-butynyl, 3-butynyl and 5-but-1-en-3-ynyl.

In the present context, “hydroxy” is intended to indicate —OH.

In the present context, “alkoxy” is intended to indicate a moiety of the formula —OR′, wherein R′ indicates alkyl as defined above. In particular “C₁₋₆alkoxy” is intended to indicate such moiety wherein the alkyl part has 1, 2, 3, 4, 5 or 6 carbon atoms. Examples of “C₁₋₆alkoxy” include methoxy, ethoxy, n-butoxy and tert-butoxy.

In the present context, “alkylsulfonyl” is intended to indicate —S(O)₂alkyl In particular C₁₋₆alkylsulfonyl is intended to indicate such a moiety wherein the alkyl part has 1, 2, 3, 4, 5 or 6 carbon atoms. Particular mention is made of methylsulfonyl.

In the present context, a “monocyclic moiety” is intended to cyclic moiety comprising only one ring, said cyclic moiety can be saturated or unsaturated.

In the present context, the terms “halo” and “halogen” are used interchangeably and refer to fluorine, chlorine, bromine or iodine.

In the present context, the term “cyano” indicates the group —C≡N, which consists of a carbon atom triple-bonded to a nitrogen atom.

In the present context, “ring atom” is intended to indicate the atoms constituting a ring, and ring atoms are selected from C, N, O and S. As an example, benzene and toluene both have 6 carbons as ring atoms whereas pyridine has 5 carbons and 1 nitrogen as ring atoms.

In the present context, “enantiomeric excess” represents the % excess of a compound in a mixture of compound enantiomers. If for example an enantiomeric excess is 90% then the ratio of the compound to its enantiomer is 95:5 and if an enantiomeric excess is 95% then the ratio of the compound to its enantiomer is 97.5:2.5. Likewise, “diastereomeric excess” represents % excess of a compound in a mixture of compound diastereomers.

In the present context, pharmaceutically acceptable salts include pharmaceutically acceptable acid addition salts, pharmaceutically acceptable metal salts, ammonium and alkylated ammonium salts. Acid addition salts include salts of inorganic acids as well as organic acids.

Examples of suitable inorganic acids include hydrochloric, hydrobromic, hydroiodic, phosphoric, sulfuric, sulfamic, nitric acids and the like.

Examples of suitable organic acids include formic, acetic, trichloroacetic, trifluoroacetic, propionic, benzoic, cinnamic, citric, fumaric, glycolic, itaconic, lactic, methanesulfonic, maleic, malic, malonic, mandelic, oxalic, picric, pyruvic, salicylic, succinic, methane sulfonic, ethanesulfonic, tartaric, ascorbic, pamoic, bismethylene salicylic, ethanedisulfonic, gluconic, citraconic, aspartic, stearic, palmitic, EDTA, glycolic, p-aminobenzoic, glutamic, benzenesulfonic, p-toluenesulfonic acids, theophylline acetic acids, as well as the 8-halotheophyllines, for example 8-bromotheophylline and the like. Further examples of pharmaceutical acceptable inorganic or organic acid addition salts include the pharmaceutically acceptable salts listed in Berge, S. M. et al., J. Pharm. Sci. 1977, 66, 2, which is incorporated herein by reference. Examples of metal salts include lithium, sodium, potassium, magnesium salts and the like. Examples of ammonium and alkylated ammonium salts include ammonium, methyl-, dimethyl-, trimethyl-, ethyl-, hydroxyethyl-, diethyl-, n-butyl-, sec-butyl-, tert-butyl-, tetramethylammonium salts and the like.

In the present context, pharmaceutical carriers include inert solid diluents or fillers, sterile aqueous solutions and various organic solvents. Examples of solid carriers include lactose, terra alba, sucrose, cyclodextrin, talc, gelatin, agar, pectin, acacia, magnesium stearate, stearic acid and lower alkyl ethers of cellulose. Examples of liquid carriers include, but are not limited to, syrup, peanut oil, olive oil, phospholipids, fatty acids, fatty acid amines, polyoxyethylene and water. Similarly, the carrier may include any sustained release material known in the art, such as glyceryl monostearate or glyceryl distearate, alone or mixed with a wax.

In the present context, the term “therapeutically effective amount” of a compound means an amount sufficient to cure, alleviate or partially arrest the clinical manifestations of a given disease and its complications in a therapeutic intervention comprising the administration of said compound. An amount adequate to accomplish this is defined as “therapeutically effective amount”. Effective amounts for each purpose will depend on the severity of the disease or injury as well as the weight and general state of the subject. It will be understood that determining an appropriate dosage may be achieved using routine experimentation, by constructing a matrix of values and testing different points in the matrix, which is all within the ordinary skills of a trained physician.

In the present context, the term “treatment” and “treating” means the management and care of a patient for the purpose of combating a condition, such as a disease or a disorder. The term is intended to include the full spectrum of treatments for a given condition from which the patient is suffering, such as administration of the active compound to alleviate the symptoms or complications, to delay the progression of the disease, disorder or condition, to alleviate or relief the symptoms and complications, and/or to cure or eliminate the disease, disorder or condition as well as to prevent the condition, wherein prevention is to be understood as the management and care of a patient for the purpose of combating the disease, condition, or disorder and includes the administration of the active compounds to prevent the onset of the symptoms or complications. In one aspect of the present invention, “treatment” and “treating” refer to prophylactic (preventive) treatment. In another aspect, “treatment” and “treating” refer to curative treatment. The patient to be treated is preferably a mammal, in particular a human being.

In the present context, the term “cognitive disorders” is intended to indicate disorders characterized by abnormalities in aspects of perception, problem solving, language, learning, working memory, memory, social recognition, attention and pre-attentional processing, such as by not limited to attention seficit hyperactivity disorder (ADHD), autism spectrum disorders, Alzheimer's disease (AD), mild cognitive impairment (MCI), age associated memory impairment (AAMI), senile dementia, vascular dementia, frontotemporal lobe dementia, Pick's disease, dementia associated with Lewy bodies, and dementia associated with Down's syndrome, cognitive impairment associated with multiple sclerosis, cognitive impairment in epilepsy, cognitive impairment associated with fragile X, cognitive impairment associated with neurofibromatosis, cognitive impairment associated with Friedreich's Ataxia, progressive supranuclear palsy (PSP), HIV associated dementia (HAD), HIV associated cognitive impairment (HIV-CI), Huntington's disease, Parkinson's disease (PD), obsessive-compulsive disorder (OCD), traumatic brain injury, epilepsy, post-traumatic stress, Wernicke-Korsakoff syndrome (WKS), post-traumatic amnesia, cognitive deficits associated with depression as well as cognitive impairment associated with schizophrenia.

The cognitive enhancing properties of a compound can be assessed e.g. by the attentional set-shifting paradigm which is an animal model allowing assessment of executive functioning via intra-dimensional (ID) versus extra-dimensional (ED) shift discrimination learning. The study can be performed by testing whether the compound is attenuating “attentional performance impairment” induced by subchronic PCP administration in rats as described by Rodefer, J. S. et al., Eur. J. Neurosci. 2005, 21:1070-1076.

In the present context, the term “autism spectrum disorders” is intended to indicate disorders characterized by widespread abnormalities of social interactions and verbal and non-verbal communication, as well as restricted interests, repetitive behavior and attention, such as by not limited to autism, Asperger syndrome, Pervasive Developmental Disorder Not Otherwise Specified (PDD-NOS), Rett syndrome, Angelmann syndrome, fragile X, DiGeorge syndrome and Childhood Disintegrative Disorder.

In the present context, the term “inflammatory disorders” is intended to indicate disorders characterized by abnormalities in the immune system such as by not limited to, allergic reactions and myopathies resulting in abnormal inflammation as well as non-immune diseases with etiological origins in inflammatory processes are thought to include by not be limited to cancer, atherosclerosis, osteoarthritis, rheumatoid arthritis and ischaemic heart disease.

### DETAILED DESCRIPTION OF THE INVENTION

The present inventors have found that certain new compounds are positive allosteric modulators (PAMs) of the α7 NNR, and as such may be used in the treatment of various disorders.

PAMs of NNRs may be dosed in combination with other drugs in order to achieve more efficacious treatment in certain patient populations. An α7 NNR PAM may act synergistically with another drug, this has been described in animals for the combination of compounds affecting nicotinic receptors, including α7 NNRs and D2 antagonism (Wiker, C., Int. J. Neuropsychopharmacol. 2008, 11(6):845-50).

Thus, compounds of the present invention may be useful treatment in the combination with another drug e.g. selected from acetylcholinesterase inhibitors, glutamate receptor antagonists, dopamine transport inhibitors, noradrenalin transport inhibitors, D2 antagonists, D2 partial agonists, PDE10 antagonists, 5-HT2A antagonists, 5-HT6 antagonists and KCNQ antagonists, lithium, sodium channel blockers, GABA signalling enhancers.

In one embodiment, compounds of the present invention are used for treatment of patients who are already in treatment with another drug selected from the list above. In one embodiment, compounds of the present invention are adapted for administration simultaneous with said other drug. In one embodiment compounds of the present invention are adapted for administration sequentially with said other drug. In one embodiment, compounds of the present invention are used as the sole medicament in treatment of a patient. In one embodiment, compounds of the present invention are used for treatment of patients who are not already in treatment with another drug selected from the list above.

### EMBODIMENTS ACCORDING TO THE INVENTION

In the following, embodiments of the invention are disclosed. The first embodiment is denoted E1, the second embodiment is denoted E2 and so forth.

E1. A compound according to formula [I]

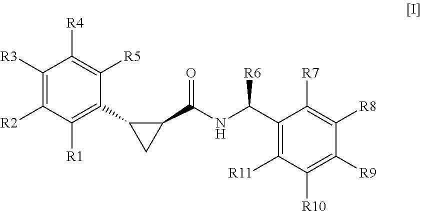

wherein R1, R2, R3, R4 and R5 are selected independently of each other from H and fluorine;
R6 is selected from methyl, methoxymethyl, hydroxymethyl and hydroxyethyl;
R7, R8, R9, R10 and R11 are selected independently of each other from H, C₁₋₆alkyl, C₂₋₆alkenyl, C₂₋₆alkynyl, C₁₋₆alkoxy, hydroxy, cyano, NR12R13, C₁₋₆alkylsulfonyl, halogen and OR14, wherein said C₁₋₆alkyl, C₂₋₆alkenyl, C₂₋₆alkynyl or C₁₋₆alkoxy is optionally substituted with one or more substituents selected from chlorine, fluorine, C₁₋₆alkoxy, cyano and NR12R13;
R12 and R13 independently represent hydrogen, C₁₋₆alkyl, C₂₋₆alkenyl and C₂₋₆alkynyl;
R14 represents a monocyclic saturated ring moiety having 4-6 ring atoms wherein one of said ring atoms is O and the other ring atoms are C;
and pharmaceutically acceptable salts thereof;
with the proviso that the compound of formula [I] is other than
(1S,2S)-2-Phenyl-cyclopropanecarboxylic acid{(R)-1-[4-(2-ethyl-butoxy)-2-methoxy-phenyl]-2-hydroxy-ethyl}-amide;
(1S,2S)-2-Phenyl-cyclopropanecarboxylic acid ((R)-2-hydroxy-1-phenyl-ethyl)-amide;
(1S,2S)-2-Phenyl-cyclopropanecarboxylic acid ((S)-1-phenyl-ethyl)-amide.
   E2. The compound according to embodiment 1, wherein four or more of R1, R2, R3, R4 and R5 are H.
   E3. The compound according to embodiment 1, wherein one of R1, R2, R3, R4 and R5 is represented by fluorine and the remaining of R1, R2, R3, R4 and R5 are represented by H.
   E4. The compound according to any of embodiments 1-3, wherein R7, R8, R9, R10 and R11 are selected independently of each other from H, C₁₋₆alkyl, C₂₋₆alkenyl, C₂₋₆alkynyl, C₁₋₆alkoxy, hydroxy, cyano, C₁₋₆alkylsulfonyl and halogen, wherein said C₁₋₆alkyl, C₂₋₆alkenyl, C₂₋₆alkynyl or C₁₋₆alkoxy is optionally substituted with one or more fluorine.
   E5. The compound according to any of embodiments 1-4, wherein R7, R8, R9, R10 and R11 are selected independently of each other from H, C₁₋₆alkoxy, hydroxy and fluorine, wherein said C₁₋₆alkoxy is optionally substituted with one or more fluorine.
   E6. The compound according to any of embodiments 1-5, wherein R7, R8, R9, R10 and R11 are selected independently of each other from H, methoxy, ethoxy, trifluoromethyxy, 2-fluoroethoxy, hydroxy and fluorine.
   E7. The compound according to any of embodiments 1-6, wherein three or more of R7, R8, R9, R10 and R11 are H.
   E8. The compound according to any of embodiments 1-7, wherein one of R7, R8, R9, R10 and R11 are selected from H, methoxy, ethoxy, trifluoromethyxy, 2-fluoroethoxy, hydroxy and fluorine and the remaining of R7, R8, R9, R10 and R11 are represented by H.
   E9. The compound according to embodiment 8, wherein R9 is selected from H, methoxy, ethoxy, trifluoromethyxy, 2-fluoroethoxy, hydroxy and fluorine and R7, R8, R10 and R11 are represented by H.
   E10. The compound according to any of embodiments 1-9, wherein R6 is methyl.
   E11. The compound according to any of embodiments 1-9, wherein R6 is methoxymethyl.
   E12. The compound according to any of embodiments 1-9, wherein R6 is hydroxymethyl.
   E13. The compound according to any of embodiments 1-9, wherein R6 is hydroxyethyl.
   E14. The compound according to embodiment 1, selected from
1: (1S,2S)-2-Phenyl-cyclopropanecarboxylic acid [(S)-1-(4-methoxy-phenyl)-ethyl]-amide;
2: (1S,2S)-2-Phenyl-cyclopropanecarboxylic acid ((S)-3-hydroxy-1-phenyl-propyl)-amide;
3: (1S,2S)-2-Phenyl-cyclopropanecarboxylic acid [(S)-1-(4-fluoro-phenyl)-ethyl]-amide;
4: (1S,2S)-2-(3-Fluoro-phenyl)-cyclopropanecarboxylic acid [(R)-2-hydroxy-1-(4-methoxy-phenyl)-ethyl]-amide;
5: (1S,2S)-2-Phenyl-cyclopropanecarboxylic acid ((S)-1-p-tolyl-ethyl)-amide;
6: (1S,2S)-2-Phenyl-cyclopropanecarboxylic acid [(S)-1-(3-fluoro-phenyl)-ethyl]-amide;
7: (1S,2S)-2-Phenyl-cyclopropanecarboxylic acid [(R)-2-hydroxy-1-(4-trifluoromethoxy-phenyl)-ethyl]-amide;
8: (1S,2S)-2-Phenyl-cyclopropanecarboxylic acid [(R)-1-(4-ethoxy-phenyl)-2-hydroxy-ethyl]-amide;
9: (1S,2S)-2-Phenyl-cyclopropanecarboxylic acid [(R)-1-(2-fluoro-4-methoxy-phenyl)-2-hydroxy-ethyl]-amide;
10: (1S,2S)-2-Phenyl-cyclopropanecarboxylic acid [(R)-2-hydroxy-1-(4-methoxy-phenyl)ethyl]-amide;
11: (1S,2S)-2-Phenyl-cyclopropanecarboxylic acid [(S)-1-(3-methoxy-phenyl)-ethyl]-amide;
12: (1S,2S)-2-Phenyl-cyclopropanecarboxylic acid [(S)-1-(2-fluoro-phenyl)-ethyl]-amide;
13: (1S,2S)-2-(4-Fluoro-phenyl)-cyclopropanecarboxylic acid [(R)-2-hydroxy-1-(4-methoxy-phenyl)-ethyl]-amide;
14: (1S,2S)-2-(3-Fluoro-phenyl)-cyclopropanecarboxylic acid [(R)-2-hydroxy-1-(4-methoxy-phenyl)-ethyl]-amide;
15: (1S,2S)-2-(2-Fluoro-phenyl)-cyclopropanecarboxylic acid [(R)-2-hydroxy-1-(4-methoxy-phenyl)-ethyl]-amide;
16: (1S,2S)-2-Phenyl-cyclopropanecarboxylic acid [(R)-2-methoxy-1-(4-methoxy-phenyl)ethyl]-amide;
   and pharmaceutically acceptable salts thereof.
   E15. The compound according to embodiment 1, selected from
17: (1S,2S)-2-Phenyl-cyclopropanecarboxylic acid [(R)-1-(4-ethoxy-phenyl)-2-methoxy-ethyl]-amide;
18: (1S,2S)-2-Phenyl-cyclopropanecarboxylic acid [(R)-2-hydroxy-1-(4-hydroxy-phenyl)ethyl]-amide;
19: (1S,2S)-2-Phenyl-cyclopropanecarboxylic acid [(R)-1-(4-hydroxy-phenyl)-2-methoxy-ethyl]-amide;
20: (1S,2S)-2-(4-Fluoro-phenyl)-cyclopropanecarboxylic acid [(R)-2-methoxy-1-(4-methoxy-phenyl)-ethyl]-amide;
21: (1S,2S)-2-(3-Fluoro-phenyl)-cyclopropanecarboxylic acid [(R)-2-methoxy-1-(4-methoxy-phenyl)-ethyl]-amide;
22: (1S,2S)-2-(4-Fluoro-phenyl)-cyclopropanecarboxylic acid [(R)-2-hydroxy-1-(4-hydroxy-phenyl)-ethyl]-amide;
23: (1S,2S)-2-(3-Fluoro-phenyl)-cyclopropanecarboxylic acid [(R)-2-hydroxy-1-(4-hydroxy-phenyl)-ethyl]-amide;
24: (1S,2S)-2-Phenyl-cyclopropanecarboxylic acid {(R)-1-[4-(2-fluoro-ethoxy)-phenyl]-2-hydroxy-ethyl}-amide;
25: (1S,2S)-2-(4-Fluoro-phenyl)-cyclopropanecarboxylic acid {(R)-1-[4-(2-fluoro-ethoxy)phenyl]-2-hydroxy-ethyl}-amide;
   and pharmaceutically acceptable salts thereof.
   E16. A compound according to any of embodiments 1-15, for use as a medicament.
   E17. A compound according to any of embodiments 1-15, for use in therapy.
   E18. A compound according to any of embodiments 1-15, for use in the treatment of a disease or disorder selected from psychosis; schizophrenia; cognitive disorders; cognitive impairment associated with schizophrenia; attention deficit hyperactivity disorder (ADHD); autism spectrum disorders, Alzheimer's disease (AD); mild cognitive impairment (MCI); age associated memory impairment (AAMI); senile dementia; AIDS dementia; Pick's disease; dementia associated with Lewy bodies; dementia associated with Down's syndrome; Huntington's disease; Parkinson's disease (PD); obsessive-compulsive disorder (OCD); traumatic brain injury; epilepsy; post-traumatic stress; Wernicke-Korsakoff syndrome (WKS); post-traumatic amnesia; cognitive deficits associated with depression; diabetes, weight control, inflammatory disorders, reduced angiogenesis; amyotrophic lateral sclerosis and pain.
   E19. The compound according to embodiment 18, wherein said a disease or disorder is selected from schizophrenia; AD; ADHD; autism spectrum disorders; PD; amyotrophic lateral sclerosis; Huntington's disease; dementia associated with Lewy bodies and pain.
   E20. The compound according to embodiment 19, wherein said disease or disorder is selected from schizophrenia; AD; ADHD and autism spectrum disorders.
   E21. The compound according to embodiment 20, wherein said disease or disorder is selected from negative and/or cognitive symptoms of schizophrenia.
   E22. The compound according to any of embodiments 1-15, for use concomitantly or sequentially with a therapeutically effective amount of a compound selected from the list consisting of acetylcholinesterase inhibitors; glutamate receptor antagonists; dopamine transport inhibitors; noradrenalin transport inhibitors; D2 antagonists; D2 partial agonists; PDE10 antagonists; 5-HT2A antagonists; 5-HT6 antagonists; KCNQ antagonists; lithium; sodium channel blockers and GABA signaling enhancers in the treatment of a disease or disorder according to any of embodiments 17-20.
   E23. A pharmaceutical composition comprising a compound according to any of embodiments 1-15, and one or more pharmaceutically acceptable carrier or excipient.
   E24. The composition according to embodiment 23, which composition additionally comprises a second compound selected from the list consisting of acetylcholinesterase inhibitors; glutamate receptor antagonists; dopamine transport inhibitors; noradrenalin transport inhibitors; D2 antagonists; D2 partial agonists; PDE10 antagonists; 5-HT2A antagonists; 5-HT6 antagonists; KCNQ antagonists; lithium; sodium channel blockers and GABA signaling enhancers.
   E25. The composition according to embodiment 24, wherein said second compound is an acetylcholinesterase inhibitor.
   E26. A kit comprising a compound according to any of embodiments 1-15, together with a second compound selected from the list consisting of acetylcholinesterase inhibitors; glutamate receptor antagonists; dopamine transport inhibitors; noradrenalin transport inhibitors; D2 antagonists; D2 partial agonists; PDE10 antagonists; 5-HT2A antagonists; 5-HT6 antagonists; KCNQ antagonists; lithium; sodium channel blockers and GABA signaling enhancers.
   E27. The kit according to embodiment 26, wherein said second compound is an acetylcholinesterase inhibitor.
   E28. A method for the treatment of a disease or disorder selected from psychosis; schizophrenia; cognitive disorders; cognitive impairment associated with schizophrenia; attention deficit hyperactivity disorder (ADHD); autism spectrum disorders, Alzheimer's disease (AD); mild cognitive impairment (MCI); age associated memory impairment (AAMI); senile dementia; AIDS dementia; Pick's disease; dementia associated with Lewy bodies; dementia associated with Down's syndrome; Huntington's disease; Parkinson's disease (PD); obsessive-compulsive disorder (OCD); traumatic brain injury; epilepsy; post-traumatic stress; Wernicke-Korsakoff syndrome (WKS); post-traumatic amnesia; cognitive deficits associated with depression; diabetes, weight control, inflammatory disorders, reduced angiogenesis; amyotrophic lateral sclerosis and pain, which method comprises the administration of a therapeutically effective amount of a compound according to any of embodiments 1-15 to a patient in need thereof.
   E29. The method according to embodiment 28, wherein said disease or disorder is selected from schizophrenia; AD; ADHD; autism spectrum disorders; PD; amyotrophic lateral sclerosis; Huntington's disease; dementia associated with Lewy bodies and pain.
   E30. The method according to embodiment 29, wherein said disease or disorder is selected from schizophrenia; AD; ADHD and autism spectrum disorders.
   E31. The method according to embodiment 30, wherein said treatment comprises the treatment of negative and/or cognitive symptoms of schizophrenia.
   E32. The method according to any of embodiments 28-31, wherein said treatment further comprises the administration of a therapeutically effective amount of a second compound selected from the list consisting of acetylcholinesterase inhibitors; glutamate receptor antagonists; dopamine transport inhibitors; noradrenalin transport inhibitors; D2 antagonists; D2 partial agonists; PDE10 antagonists; 5-HT2A antagonists; 5-HT6 antagonists; KCNQ antagonists; lithium; sodium channel blockers and GABA signaling enhancers.
   E33. The method according to embodiment 32, wherein said second compound is an acetylcholinesterase inhibitor.
   E34. Use of a compound according to any of embodiments 1-15, for the manufacture of a medicament for the treatment of a disease or disorder selected from psychosis; schizophrenia; cognitive disorders; cognitive impairment associated with schizophrenia; attention deficit hyperactivity disorder (ADHD); autism spectrum disorders, Alzheimer's disease (AD); mild cognitive impairment (MCI); age associated memory impairment (AAMI); senile dementia; AIDS dementia; Pick's disease; dementia associated with Lewy bodies; dementia associated with Down's syndrome; Huntington's disease; Parkinson's disease (PD); obsessive-compulsive disorder (OCD); traumatic brain injury; epilepsy; post-traumatic stress; Wernicke-Korsakoff syndrome (WKS); post-traumatic amnesia; cognitive deficits associated with depression; diabetes, weight control, inflammatory disorders, reduced angiogenesis; amyotrophic lateral sclerosis and pain.
   E35. The use according to embodiment 34, wherein said disease or disorder is selected from schizophrenia; AD; ADHD; autism spectrum disorders; PD; amyotrophic lateral sclerosis; Huntington's disease; dementia associated with Lewy bodies and pain.
   E36. The use according to embodiment 35, wherein said disease or disorder is selected from schizophrenia; AD; ADHD and autism spectrum disorders.
   E37. The use according to embodiment 36, wherein said disease is the positive, negative and/or cognitive symptoms of schizophrenia.

The compounds of the invention may exist in unsolvated as well as in solvated forms in which the solvent molecules are selected from pharmaceutically acceptable solvents such as water, ethanol and the like. In general, such solvated forms are considered equivalent to the unsolvated forms for the purposes of this invention.

The compounds of the present invention have three asymmetric centers with fixed stereochemistry indicated by the arrows below.

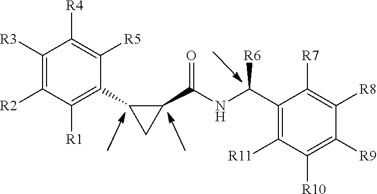

The compounds of the present invention are manufactured from two chiral intermediates with one and two asymmetric centers, respectively, as illustrated by the examples below. In this context is understood that when specifying the enantiomeric form of the intermediate, then the intermediate is in enantiomeric excess, e.g. essentially in a pure, mono-enantiomeric form. Accordingly, the resulting compounds of the invention are having a diastereomeric excess of at least 80%. One embodiment of the invention relates to a compound of the invention having a diastereomeric excess of at least 80% such as at least 85%, such as at least 90%, preferably at least 95% or at least 97% with reference to the three assymetric centers indicated above.

Dependent on the individually substituents R1-R14, the compounds of the present invention may furthermore have one or more additional asymmetric centers. It is intended that any optical isomers (i.e. enantiomers or diastereomers), in the form of separated, pure or partially purified optical isomers and any mixtures thereof including racemic mixtures, i.e. a mixture of stereoisomers, which have emerged because of asymmetric centers in any of substituents R1-R14, are included within the scope of the invention.

Racemic forms can be resolved into the optical antipodes by known methods, for example by separation of diastereomeric salts thereof with an optically active acid, and liberating the optically active amine compound by treatment with a base. Another method for resolving racemates into the optical antipodes is based upon chromatography of an optically active matrix.

The compounds of the present invention may also be resolved by the formation of diastereomeric derivatives. Additional methods for the resolution of optical isomers, known to those skilled in the art, may be used. Such methods include those discussed by J. Jaques, A. Collet and S. Wilen in “Enantiomers, Racemates, and Resolutions”, John Wiley and Sons, New York (1981). Optically active compounds can also be prepared from optically active starting materials.

Furthermore, when a double bond or a fully or partially saturated ring system is present in the molecule geometric isomers may be formed. It is intended that any geometric isomers, as separated, pure or partially purified geometric isomers or mixtures thereof are included within the scope of the invention. Likewise, molecules having a bond with restricted rotation may form geometric isomers. These are also intended to be included within the scope of the present invention.

Furthermore, some of the compounds of the present invention may exist in different tautomeric forms and it is intended that any tautomeric forms that the compounds are able to form are included within the scope of the present invention.

The compounds of the present invention may be administered alone as a pure compound or in combination with pharmaceutically acceptable carriers or excipients, in either single or multiple doses. The pharmaceutical compositions according to the invention may be formulated with pharmaceutically acceptable carriers or diluents as well as any other known adjuvants and excipients in accordance with conventional techniques such as those disclosed in Remington: The Science and Practice of Pharmacy, 19 Edition, Gennaro, Ed., Mack Publishing Co., Easton, Pa., 1995.

The pharmaceutical compositions may be specifically formulated for administration by any suitable route such as the oral, rectal, nasal, pulmonary, topical (including buccal and sublingual), transdermal, intracisternal, intraperitoneal, vaginal and parenteral (including subcutaneous, intramuscular, intrathecal, intravenous and intradermal) route, the oral route being preferred. It will be appreciated that the preferred route will depend on the general condition and age of the subject to be treated, the nature of the condition to be treated and the active ingredient chosen.

Pharmaceutical compositions for oral administration include solid dosage forms such as capsules, tablets, dragees, pills, lozenges, powders and granules. Where appropriate, they can be prepared with coatings.

Liquid dosage forms for oral administration include solutions, emulsions, suspensions, syrups and elixirs.

Pharmaceutical compositions for parenteral administration include sterile aqueous and nonaqueous injectable solutions, dispersions, suspensions or emulsions as well as sterile powders to be reconstituted in sterile injectable solutions or dispersions prior to use. Other suitable administration forms include suppositories, sprays, ointments, cremes, gels, inhalants, dermal patches, implants, etc.

In one embodiment, the compound of the present invention is administered in an amount from about 0.001 mg/kg body weight to about 100 mg/kg body weight per day. In particular, daily dosages may be in the range of 0.01 mg/kg body weight to about 50 mg/kg body weight per day. The exact dosages will depend upon the frequency and mode of administration, the sex, the age the weight, and the general condition of the subject to be treated, the nature and the severity of the condition to be treated, any concomitant diseases to be treated, the desired effect of the treatment and other factors known to those skilled in the art.

A typical oral dosage for adults will be in the range of 0.1-1000 mg/day of a compound of the present invention, such as 1-500 mg/day, such as 1-100 mg/day or 1-50 mg/day. Conveniently, the compounds of the invention are administered in a unit dosage form containing said compounds in an amount of about 0.1 to 500 mg, such as 10 mg, 50 mg 100 mg, 150 mg, 200 mg or 250 mg of a compound of the present invention.

For parenteral administration, solutions of the compound of the invention in sterile aqueous solution, aqueous propylene glycol, aqueous vitamin E or sesame or peanut oil may be employed. Such aqueous solutions should be suitably buffered if necessary and the liquid diluent first rendered isotonic with sufficient saline or glucose. The aqueous solutions are particularly suitable for intravenous, intramuscular, subcutaneous and intraperitoneal administration. The sterile aqueous media employed are all readily available by standard techniques known to those skilled in the art.

Suitable pharmaceutical carriers include inert solid diluents or fillers, sterile aqueous solution and various organic solvents. Examples of solid carriers are lactose, terra alba, sucrose, cyclodextrin, talc, gelatine, agar, pectin, acacia, magnesium stearate, stearic acid and lower alkyl ethers of cellulose. Examples of liquid carriers are syrup, peanut oil, olive oil, phospho lipids, fatty acids, fatty acid amines, polyoxyethylene and water. The pharmaceutical compositions formed by combining the compound of the invention and the pharmaceutical acceptable carriers are then readily administered in a variety of dosage forms suitable for the disclosed routes of administration.

Formulations of the present invention suitable for oral administration may be presented as discrete units such as capsules or tablets, each containing a predetermined amount of the active ingredient, and which may include a suitable excipient. Furthermore, the orally available formulations may be in the form of a powder or granules, a solution or suspension in an aqueous or non-aqueous liquid, or an oil-in-water or water-in-oil liquid emulsion. If a solid carrier is used for oral administration, the preparation may be tablet, e.g. placed in a hard gelatine capsule in powder or pellet form or in the form of a troche or lozenge. The amount of solid carrier may vary but will usually be from about 25 mg to about 1 g. If a liquid carrier is used, the preparation may be in the form of a syrup, emulsion, soft gelatine capsule or sterile injectable liquid such as an aqueous or non-aqueous liquid suspension or solution.

Tablets may be prepared by mixing the active ingredient with ordinary adjuvants and/or diluents followed by the compression of the mixture in a conventional tabletting machine. Examples of adjuvants or diluents comprise: Corn starch, potato starch, talcum, magnesium stearate, gelatine, lactose, gums, and the like. Any other adjuvants or additives usually used for such purposes such as colourings, flavourings, preservatives etc. may be used provided that they are compatible with the active ingredients.

All references, including publications, patent applications, and patents, cited herein are hereby incorporated by reference in their entirety and to the same extent as if each reference were individually and specifically indicated to be incorporated by reference and were set forth in its entirety herein (to the maximum extent permitted by law), regardless of any separately provided incorporation of particular documents made elsewhere herein. The use of the terms “a” and “an” and “the” and similar referents in the context of describing the invention are to be construed to cover both the singular and the plural, unless otherwise indicated herein or clearly contradicted by context. For example, the phrase “the compound” is to be understood as referring to various “compounds” of the invention or particular described aspect, unless otherwise indicated.

The description herein of any aspect or aspect of the invention using terms such as “comprising”, “having,” “including,” or “containing” with reference to an element or elements is intended to provide support for a similar aspect or aspect of the invention that “consists of”, “consists essentially of”, or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context). It should be understood that the various aspects, embodiments, implementations and features of the invention mentioned herein may be claimed separately, or in any combination.

The compounds of formula I may be prepared by methods described below, together with synthetic methods known in the art of organic chemistry, or modifications that are familiar to those of ordinary skill in the art. The starting materials used herein are available commercially or may be prepared by routine methods known in the art, such as those method described in standard reference books such as “Compendium of Organic Synthetic Methods, Vol. I-XII” (published with Wiley-Interscience). Preferred methods include, but are not limited to, those described below.

The schemes are representative of methods useful in synthesizing the compounds of the present invention. They are not to constrain the scope of the invention in any way.

### Methods of Preparation of the Compounds of the Invention.

The compounds of the invention with formula [I] can be prepared from intermediate III and II as described in Scheme 1.

### General Synthetic Schemes

The compounds of formula [I] may be prepared by methods described below, together with synthetic methods known in the art of organic chemistry, or modifications that are familiar to those of ordinary skill in the art. The starting materials used herein are available commercially or may be prepared by routine methods known in the art, such as those method described in standard reference books such as “Compendium of Organic Synthetic Methods, Vol. I-XII” (published with Wiley-Interscience). Preferred methods include, but are not limited to, those described below.

The schemes are representative of methods useful in synthesizing the compounds of the present invention. They are not to constrain the scope of the invention in any way.

It is understood that when typical or preferred reagents and experimental conditions are used (e.g. equivalents, solvents, temperatures, reaction times etc.) alternative experimental conditions can also be used—unless otherwise stated. The optimum reaction conditions may vary with specific reactants and experimental conditions, but can be optimized by a person skilled in the art by using routine optimization approaches.

### Methods of Preparation of the Compounds of the Invention.

The compounds of the invention with formula I can be prepared from intermediate III and II as described in Scheme 1.

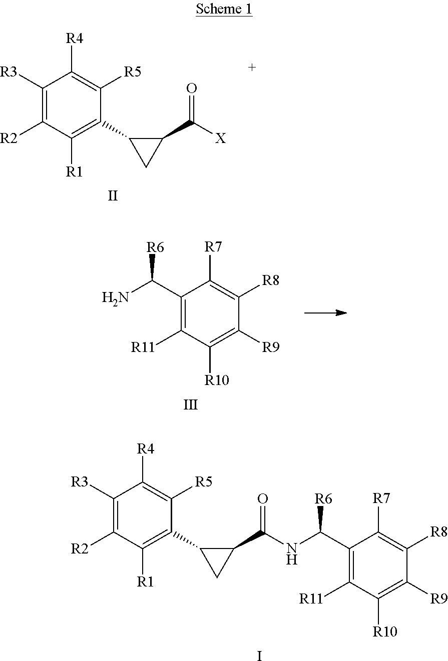

If X is a hydroxyl, the carboxylic acid II and the amine III can be condensed to form the amide I using standard peptide coupling chemistry, e.g. as described in the textbook Synthetic Peptides A user's Guide (Edited by Gregory A. Grant, W. H. Freeman and company (1992) ISBN 0-7167-7009-1) or as described in the textbook Houben-Weyl Volume E22a Synthesis of peptides (George Thiemes Verlag Stuttgart (2003) 4^{th} ed.). One example of this amide formation is the use of the coupling reagent HATU (O-(benzotriazol-1-yl)-N,N,N′,N′-tetramethyluronium hexafluorophosphate). Typically, one eq. of II is reacted with one eq. of HATU in the presence of two eq. of a tertiary amine e.g. triethylamine in a suitable solvent e.g. DMF. After a short period of time (e.g. five minutes) this mixture is reacted with one eq. of III to form I. Another example of this amide formation uses 1-hydroxybenzotriazole together with the water soluble carbodiimide EDC (CAS 25952-53-8) and triethyl amine in a suitable solvent e.g. THF. These reactions are usually performed at room temperature or between 0° C. and 50° C.

If X is a chloride (e.g. prepared from the carboxylic acid II, X=OH, using thionyl chloride) Ill can be reacted with II to form I in the presence of a tertiary amine in a suitable solvent. Alternatively, the carboxylic acid chloride (II, X=Cl) can be reacted with N-hydroxy succinimide to produce the HOSU ester which can be isolated and then reacted with III to produce I.

### Methods of Preparation of the Intermediates of the Invention.

The Intermediates of the invention with formula II are either commercially available or can be prepared as described in Scheme 2.

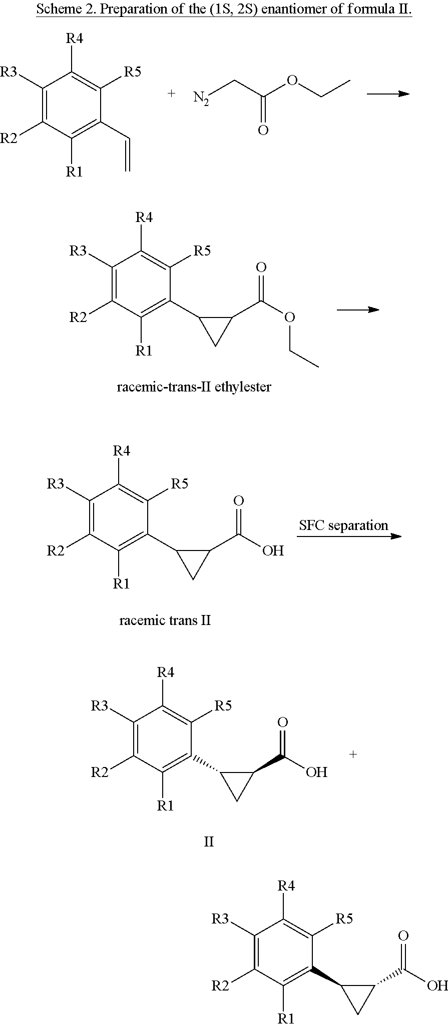

Ethyldiazoacetate can be reacted with the styrene in Scheme II to produce the racemic-trans II ethyl ester. This ester can then be hydrolyzed to racemic trans II which can then be separated into the two enantiomers using SFC. Alternatively, racemic trans II can be resolved into the two enantiomers by known methods as described in the textbook “Enantiomers, Racemates and Resolutions” (J. Jaques, et al., John Wiley and sons, New York (1981)).

Another preparation of the compounds with formula II is described in Scheme 3. This method has been described in detail in WO2012/037258

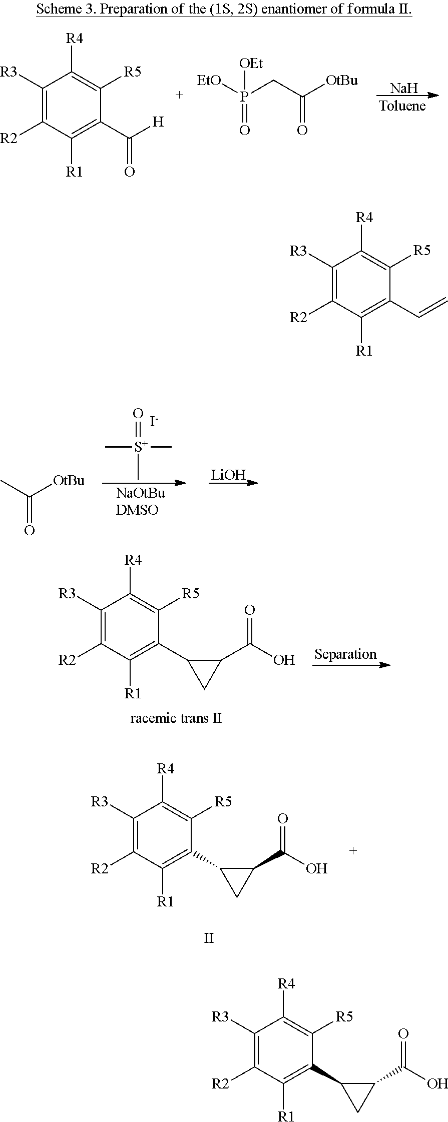

The benzaldehyde shown in Scheme 3 can be reacted with the anion of (Diethoxyphosphoryl)-acetic acid tert-butyl ester to produce the unsaturated ester shown. Cylopropanation followed by hydrolysis then produces Racemic trans II, which can be separated as described above.

The Intermediates of the invention with formula III are either commercially available or can be prepared as described in Scheme 4 in which R₆ is CH₂OH.

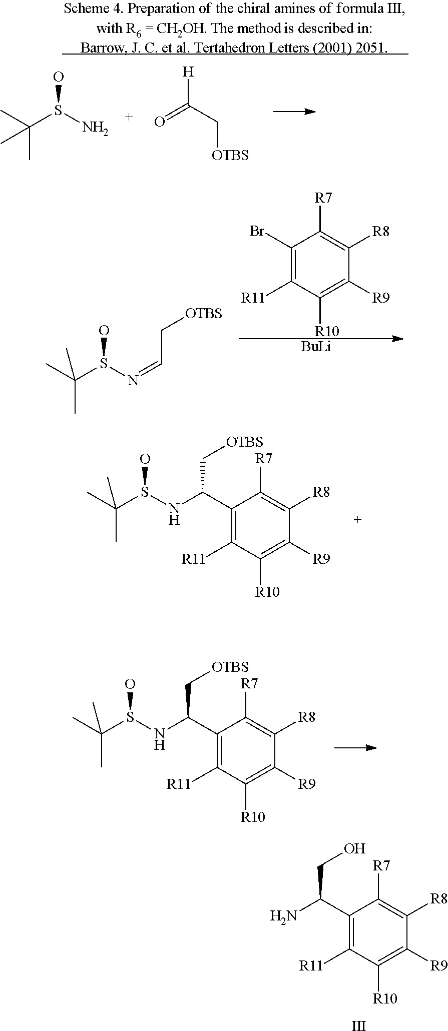

(R)-(+)-2-methyl-2-propanesulfinamide can be reacted with (tertbutyldimethylsilyloxy)acetaldehyde as described in the literature (Barrow, J. C. et al. Tetrahedron Letters (2001) 2051) to produce the sulfinimine shown in Scheme 4. 1,2-addition of an organometallic (e.g. a Grignard reagent or an aryllithiumreagent (shown in Scheme 4) reagent to this sulfinyl imines then gives the two diastereomeric protected amino alcohols shown in scheme 4. These isomers can be separated e.g. by silica gel chromatography and the protecting groups are then removed under acidic conditions.

Another method using enantiopure tert-butanesulfinamide is shown in Scheme 5 (Robak, M., Herbage, M., Ellman, Chem. Rev. 2010, 110, 3600-3740 and references cited herein). For simplicity, the method is only illustrated for R₆=CH₃, but the method is not limited to R₆=CH₃.

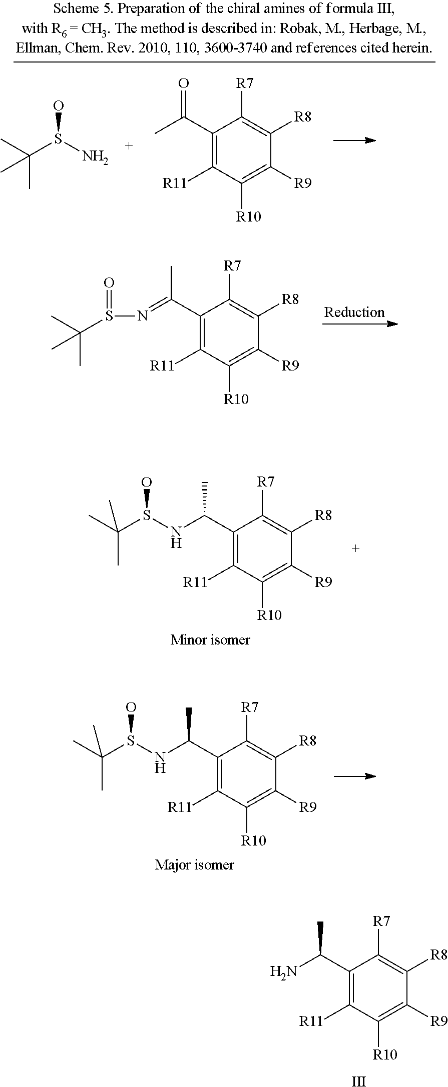

(R)-(+)-2-methyl-2-propanesulfinamide can be reacted with a suitable ketone and titanium(IV)ethoxide in a suitable solvent e.g. THF under heating conditions to produce the sulfinyl imine shown in scheme 5. This imine can be reduced, with some selectivity using a reducing agent (e.g. L-selectride) in a suitable solvent (e.g. THF) at a suitable temperature (e.g. −70° C.) to produce the major and the minor isomer shown in Scheme 5. The major isomer can be isolated by e.g. silica gel chromatography and the chiral auxiliary can then be removed with acid (e.g. HCl in water) to produce III.

### EXAMPLES

The invention will be illustrated by the following non-limiting examples. Chemical names were obtained using the software MDL ISIS/DRAW 2.5 from MDL information systems.

### ABBREVIATIONS

α_{D}=specific optical rotation. Boc₂O=Boc anhydride/di-t-butyl dicarbonate (e.g. Aldrich 19, 913-3). Brine=saturated aqueous solution of sodium chloride. CDCl₃ deuterated chloroform (e.g. Aldrich 225789). Celite=filter-aid. DMF=dimethyl formamide. DMSO=dimethyl sulfoxide. Et₃N=triethyl amine. EtOAc=ethyl acetate. 99% EtOH=absolute ethanol. h=hours. HATU=O-(7-Azabenzotriazol-1-yl)-N,N,N′,N′-tetramethyluronium hexaflouruphosphate. LC-MS=high-performance liquid chromatography/mass spectrometer. MeOH=methanol. min=minutes. NaH=sodium hydride (used as a 60% dispersion; Aldrich 45, 291-2). NaOH=aqueous solution of sodium hydroxide. sat. NaHCO₃=saturated aqueous solution of sodium hydrogen carbonate. SFC=supercritical flash chromatography. THF=tetrahydrofuran (dried over 4 Å molecular sieves). EDCI=1-ethyl-3-(3-dimethylaminopropyl)carbodiimide hydrochloride. PE=Petroleum ether.

Chemical names were obtained using the software MDL ISIS/DRAW 2.5 from MDL information systems

### Spectroscopic Methods

### LC-MS:

LC-MS were run on Waters Aquity UPLC-MS consisting of Waters Aquity including column mamager, binary solvent manager, sample organizer, PDA detector (operating at 254 nM), ELS detector, and SQ-MS equipped with APPI-source operating in positive ion mode. LC-conditions: The column was Acquity UPLC BEH C18 1.7 μm; 2.1×50 mm operating at 60° C. with 1.2 mL/min of a binary gradient consisting of water+0.1% formic acid (A) and acetonitrile+5% water+0.1% formic acid.

| | | |
|---|---|---|
| Gradient: | 0.00 min | 10% B |
| | 1.00 min | 99.9% B |
| | 1.01 min | 10% B |
| | 1.15 min | 10% B |

Total run time: 1.15 min

### Method A:

Preparative supercritical fluid chromatography (SFC) was performed on a Berger Multigram II operating at 50 mL/min at 35° C. and 100 bar backpressure using stacked injections. The column was a ChiralpakAD 5 u, 250×21 mm. The eluent was CO₂ (70%) and ethanol (30%).

### Method B:

Performed as outlined above. The column was a Chiral OJ 250×30 mm. The eluent was CO₂ (80%) and MeOH (20%).

### Method C:

Preparative supercritical fluid chromatography (SFC) was performed on a Thar SFC-80 operating at 60 g/min at 35° C. and 140 bar backpressure using stacked injections. The column was a ChiralPakAD-H (250×30 mm). The eluent was CO₂ (88%) and Ethanol (12%).

### Method D:

Preparative supercritical fluid chromatography (SFC) was performed on a Thar SFC-200 operating at 100 g/min at 35° C. and 140 bar backpressure using stacked injections. The column was a ChiralPakAD-H (250×30 mm). The eluent was CO₂ (90%) and Ethanol (10%).

### Method E:

Preparative HPLC was performed on a Gilson GX281 instrument equipped with a Gemini column. Mobile phase A water. Mobile phase B: acetonitrile. Column temperature: 30° C. Gradient: 35-60% B 0-25 min. Flow rate: 80 mL/min.

### Method F:

Preparative HPLC was performed on a Gilson GX281 instrument equipped with a Gemini column. Mobile phase A water (containing 0.03% NH₃) Mobile phase B: acetonitrile. Column temperature: 30° C. Gradient: 35-65% B 0-10 min. Flow rate: 25 mL/min.

### Preparation of Intermediates

### IM1: (1S,2S)-2-Phenyl-cyclopropanecarboxylic acid

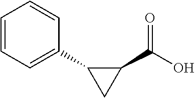 [SMILES: O=C(O)[C@H]1C[C@@H]1c1ccccc1]

Commercially available, racemic trans 2-phenyl-cyclopropanecarboxylic acid (Sigma-Aldrich, catalog no P22354) was subjected to chiral SFC separation, method A to give IM1 as an oil that slowly solidified upon standing. Specific optical rotation +300.9° [α]_{D}²⁰ (C=1% EtOH). (Lit: +389° [α]_{D}²⁰ (C=0.61, CHCl₃) Kozikowski et al., J. Med. Chem. 2009, 52, 1885-1902), (Lit: +311.7° [α]_{D}²⁰ (C=1.776, EtOH) Walborsky et al., Tetrahedron 1964, 20, 1695-1699.)

### IM2: (1S,2S)-2-(2-Fluoro-phenyl)-cyclopropanecarboxylic acid

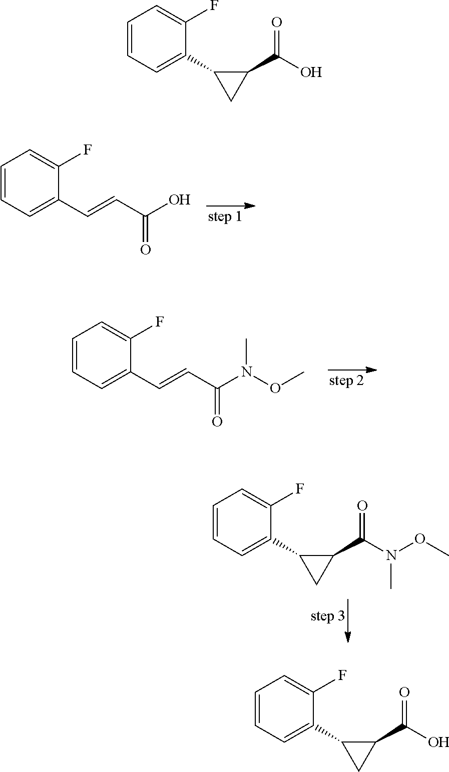 [SMILES: O=C(O)[C@H]1C[C@@H]1c1ccccc1F ; O=C(O)/C=C/c1ccccc1F ; CON(C)C(=O)/C=C/c1ccccc1F ; CON(C)C(=O)[C@H]1C[C@@H]1c1ccccc1F ; O=C(O)[C@H]1C[C@@H]1c1ccccc1F]

### Step 1:

To a solution of (E)-3-(2-Fluoro-phenyl)-acrylic acid (2.0 g, 12 mmol), EDCI (2.8 g, 14.4 mmol), DMAP (1.5 g, 12 mmol) and Et₃N (2.4 g, 24 mmol) in 50 mL of methylene chloride was added O,N-dimethyl-hydroxylamine hydrochloride (1.4 g, 14.4 mol). The reaction was kept at room temperature overnight. The mixture was quenched by water and extracted with methylene chloride (100 mL). The combined organic layer was dried over Na₂SO₄ and evaporated to dryness. Flash chromatography on (silica, petroleum ether:EtOAc=3:1) gave 3-(2-fluoro-phenyl)-N-methoxy-N-methyl-acrylamide as a colorless liquid (2.21 g, 88%). 1H NMR (CDCl₃) δ 7.80-7.84 (m, 1H), 7.53-7.58 (m, 1H), 7.30-7.35 (m, 1H), 7.06-7.17 (m, 3H), 3.76 (s, 3H), 3.31 (s, 3H).

### Step 2:

To a mixture of NaH (0.43 g, 17.8 mmol) in DMF (10 mL) was added a solution of trimethylsulfonium iodide (3.9 g, 17.8 mmol) in DMF (10 mL) dropwise at 0° C. over 30 minutes. The reaction was kept at room temperature for 30 minutes under N₂. The mixture was cooled to 0° C. A solution of 3-(2-fluoro-phenyl)-N-methoxy-N-methyl-acrylamide (1.9 g, 8.9 mmol) in DMF (10 mL) was then added and the resulting mixture was kept for 2 h without cooling. The mixture was quenched by water, concentrated and extracted with methylene chloride (50 mL). The combined organic layer was dried over Na₂SO₄ and evaporated to dryness. Flash chromatography (silica, petroleum ether:EtOAc=3:1) gave trans-2-(2-fluoro-phenyl)cyclopropanecarboxylic acid methoxy-methyl-amide as a yellow solid (1.8 g, 90%). 1H NMR (CDCl₃) δ 7.14-7.26 (m, 1H), 6.97-7.07 (m, 3H), 3.71 (s, 3H), 3.24 (s, 3H), 2.59-2.64 (m, 1H), 2.43 (s, 1H), 1.59-1.64 (m, 1H), 1.31-1.36 (m, 1H).

### Step 3:

To a solution of trans-2-(2-fluoro-phenyl)-cyclopropanecarboxylic acid methoxy-methyl-amide (1.8 g, 8.5 mmol) in MeOH/H₂O (20 mL/4 mL) was added NaOH (0.7 mg, 17 mmol). The resulting mixture was heated to reflux for 3 h. The volatiles were removed in vacuo. The mixture was washed with EtOAc (100 mL). The organic layer was extracted with H₂O (100 mL). The combined aqueous layer was acidified with 3 N HCl until pH=1-2. The mixture was ectracted with EtOAc (100 mL). The organic layer was dried over Na₂SO₄ and evaporated to dryness. Separation of the enantiomers by preparative SFC (Method B) gave the title compound IM2 as a solid (446 mg, 30%) as a solid. 1H NMR (CDCl₃) δ 7.17-7.22 (m, 1H), 6.96-7.08 (m, 3H), 2.72-2.77 (m, 1H), 1.92-1.96 (m, 1H), 1.64-1.69 (m, 1H), 1.42-1.47 (m, 1H). [α]_{D}²⁰=+223.0 (c=0.1, MeOH).

### IM3: (1S,2S)-2-(3-Fluoro-phenyl)-cyclopropanecarboxylic acid

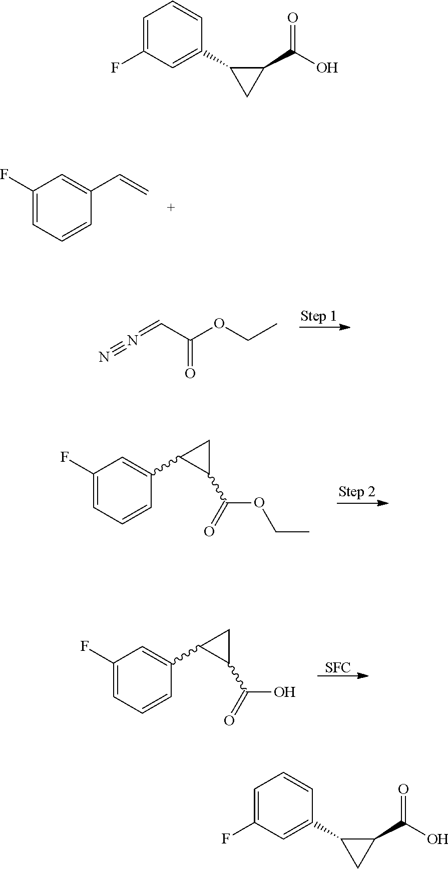

### Step 1:

A round-bottomed flask was charged with 3-fluorostyrene (13.0 g, 0.107 mol) in anhydrous methylene chloride (130 mL). To this mixture was added rhodium acetate dimer (1.30 g, cat amount). A solution of ethyldiazoacetate (33.28 g, 0.291 mol) in anhydrous methylene chloride (130 mL) was added to the reaction via a syringe pump over 5 h and stirred at room temperature for 1 h in darkness. The reaction mixture was filtered through a plug of celite, which was washed with water followed by brine. The organic layer was dried over Na₂SO₄ and evaporated to dryness. Flash chromatography (silica, EtOAc/petroleum ether 1:9) gave rac-trans 2-(3-fluoro-phenyl)-cyclopropanecarboxylic acid ethyl ester (13.0 g, 59%) as a colorless liquid sufficiently pure for the next step.

### Step 2:

To a solution of rac-trans 2-(3-fluoro-phenyl)-cyclopropanecarboxylic acid ethyl ester (13.0 g, 0.062 mol) in MeOH (310 mL) was added a solution of KOH (35.0 g, 0.625 mol) in MeOH (150 mL) at 0° C. After addition of the base the reaction mixture was stirred at room temperature for 18 h. The reaction mixture was poured into water and extracted with methylene chloride (2×50 mL). The aqueous layer was acidified with 10% HCl. The resulting mixture was extracted with methylene chloride (2×150 mL). The combined organic layers were dried over Na₂SO₄ and evaporated to dryness to give rac-trans-2-(3-fluoro-phenyl)cyclopropanecarboxylic acid as colorless crystals (9.5 g, 85%). Separation of the isomers by chiral SFC (Method C) gave the title compound (1S,2S)-2-(3-fluoro-phenyl)-cyclopropanecarboxylic acid IM2 as colorless crystals (3.27 g, 17% overall yield from 3-fluorostyrene) sufficiently pure for the next step. Specific optical rotation +263.4° [α]_{D}²⁰ (C=1% MeOH)

### IM4: (1S,2S)-2-(4-Fluoro-phenyl)-cyclopropanecarboxylic acid

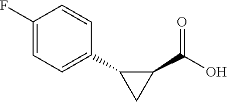 [SMILES: O=C(O)[C@H]1C[C@@H]1c1ccc(F)cc1]

Prepared analogously to IM2 using SFC (Method D) to give the title compound sufficiently pure for the next step (3.1 g, 13% overall yield from 4-fluorostyrene). Specific optical rotation +263.2° [α]_{D}²⁰ (C=1% MeOH)

### IM5: (R)-2-Methyl-propane-2-sulfinic acid [2-methoxy-eth-(E)-ylidene]-amide

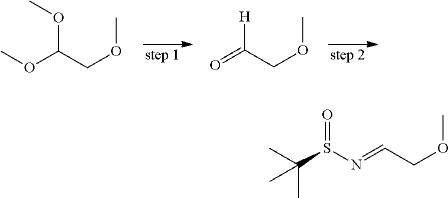

### Step 1:

To a solution of 1,1,2-Trimethoxy-ethane (15 g, 0.125 mol) in THF (100 mL) was added HCl (10 mL, 12 N aq.) at room temperature. The reaction mixture was stirred at reflux overnight. The mixture was dried over anhydrous Na₂SO₄, then Na₂CO₃, filtered through celite, and washed with DCM (100 mL×2). This solution of crude methoxy-acetaldehyde was used in step 2 without purification

### Step 2:

To the solution of methoxy-acetaldehyde (0.125 mol) prepared above, was added (R)-tert-butanesulfinamide (15.1 g, 0.125 mol) and anhydrous CuSO₄ (40 g, 0.25 mol) in DCM (250 mL) and the mixture was stirred at room temperature overnight. The mixture was filtered through celite and the filter cake was washed with DCM (100 mL×3). The combined filtrate was evaporated in vacuum, and purified via silica gel chromatography (eluted: petroleum ether:EtOAc from 10:1 to 2:1) to afford (R)-2-Methyl-propane-2-sulfinic acid [2-methoxy-eth(E)-ylidene]-amide (4.27 g, yield: 12% based on compound 1) as a colorless oil.

### IM6: (R)-1-(4-Ethoxy-phenyl)-2-methoxy-ethylamine hydrochloride

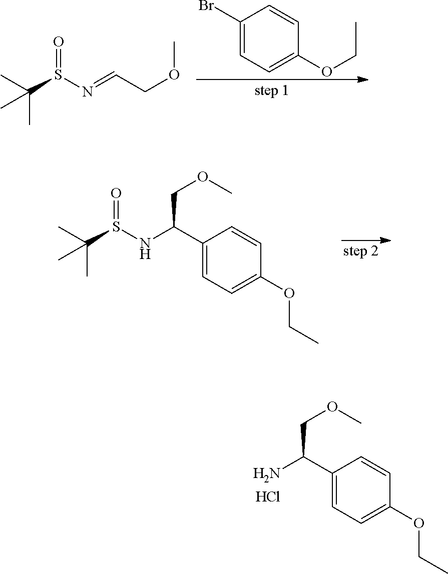

### Step 1:

To a solution of 1-Bromo-4-ethoxy-benzene (2.0 g, 10 mmol) in THF (50 mL) at −78° C. was added n-BuLi (4 mL, 10 mmol) over 15 min. After addition, the reaction mixture was stirred for 30 min at −78° C. Then a solution of IM5 (1.5 g, 8.47 mol) in THF (20 mL) was added dropwise at −78° C. The reaction mixture was stirred for 2 h at −78° C., then 2 hours at room temperature. The solution was quenched with H₂O (50 mL), extracted with MTBE (50 mL×2). The combined organic layers were dried over Na₂SO₄, filtered and concentrated in vacuum. The residue was purified by silica gel chromatography (eluted: PE:EtOAc=5:1 to 1:1) to afford a mixture of isomers (0.5 g, yield: 20%, isomer ratio=85:15). The isomer mixture was separated by SFC (modified Method C: column temp 38° C. and Nozzle pressure 100 bar) (isolating the faster fraction) to afford (R)-2-Methyl-propane-2-sulfinic acid [(R)-1-(4-ethoxy-phenyl)-2-methoxy-ethyl]-amide (310 mg) as a yellow oil. ¹H NMR (CDCl₃): δ 7.24 (d, J=8.8 Hz, 2H), 6.86 (d, J=8.8 Hz, 2H), 4.58-4.63 (m, 1H), 4.08 (s, 1H), 4.02 (q, J=7.2 Hz, 2H), 3.44-3.54 (m, 2H), 3.39 (s, 3H), 1.41 (t, J=7.2 Hz, 3H), 1.21 (s, 9H). [α]_{D}²⁰=−146.6° (c=0.1, MeOH).

### Step 2:

To a solution of (R)-2-Methyl-propane-2-sulfinic acid [(R)-1-(4-ethoxy-phenyl)-2-methoxy-ethyl]-amide (280 mg, 0.94 mmol) in anhydrous dioxane (5 mL) at 0° C. was added HCl/dioxane (5 mL, 4.0 M) and the reaction was stirred at 0° C. for 30 min. Diluted with MTBE (50 mL), the white precipitate was collected by filtration, dried to afford IM6 (230 mg, yield: 100%) as a white solid. ¹H NMR (DMSO-d₆): δ 8.49 (s-broad, 3H), 7.39-7.43 (m, 2H), 6.94-6.99 (m, 2H), 4.37-4.43 (m, 1H), 4.02 (q, J=7.2 Hz, 2H), 3.63-3.68 (m, 1H), 3.57 (s, 3H), 3.53-3.57 (m, 1H), 1.31 (t, J=7.2 Hz, 3H).

### IM7: 4-((R)-1-Amino-2-methoxy-ethyl)-phenol hydrochloride

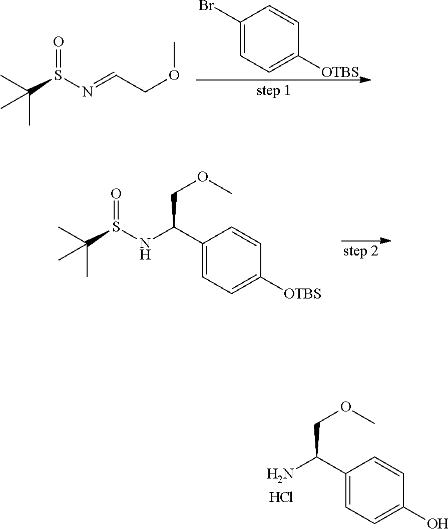 [SMILES: COC/C=N/[S@](=O)C(C)(C)C ; CC(C)(C)[Si](C)(C)Oc1ccc(Br)cc1 ; COC[C@H](N[S@](=O)C(C)(C)C)c1ccc(O[Si](C)(C)C(C)(C)C)cc1 ; Cl ; COC[C@H](N)c1ccc(O)cc1]

### Step 1:

To a solution of (4-Bromophenoxy)-tert-butyldimethylsilane (Sigma-Aldrich catalogue nr 444774) (2.75 g, 9.58 mmol) in THF (20 mL) at −78° C. was added t-BuLi (20 mL, 20 mmol) over 20 min. After addition, the reaction mixture was stirred for 30 min at −78° C. Then a solution of IM5 (1.5 g, 8.47 mol) in THF (10 mL) was added dropwise at −78° C. The reaction mixture was stirred for 2 h at −78° C., then 1 hour at room temperature. The solution was quenched with H₂O (50 mL), extracted with EtOAc (50 mL×3). The combined organic layers were dried over Na₂SO₄, filtered and concentrated in vacuum. The residue was purified by silica gel chromatography (eluted: PE:EtOAc=10:1 to 2:1) to afford isomer mixture (1.2 g, isomer ratio=85:15). The isomer mixture was separated by SFC (modified Method C: column temp 38° C. and Nozzle pressure 100 bar) (isolating the faster fraction) to afford a TBS protected intermediate (310 mg; yield: 36.8%) as yellow oil. ¹H NMR (CDCl₃): δ 7.18 (d, J=6.4 Hz, 2H), 6.79 (d, J=6.4 Hz, 2H), 4.57-4.62 (m, 1H), 4.05 (s, 1H), 3.44-3.54 (m, 2H), 3.39 (s, 3H), 1.21 (s, 9H), 0.97 (s, 9H), 0.19 (s, 6H). [α]_{D}²⁰=−90.0° (c=0.1, MeOH).

### Step 2:

A solution of this intermediate (385 mg, 1.0 mmol) in HCl/dioxane (5 mL, 4.0 M) was stirred at 40° C. overnight. Diluted with MTBE (50 mL), the white precipitate was collected by filtration, dried to afford IM7 (203 mg, yield: 100%) as a white solid which was used for next step without further purification.

### IM8: (R)-2-Methoxy-1-(4-methoxy-phenyl)-ethylamine hydrochloride

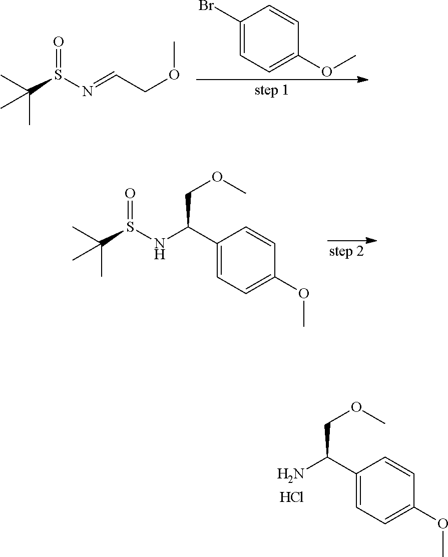

### Step 1:

To a solution of 1-Bromo-4-methoxy-benzene (4.8 g, 25.7 mmol) in THF (50 mL) at −78° C. was added n-BuLi (11.2 mL, 28 mmol). After addition, the reaction mixture was stirred for 30 minutes at −78° C. Then a solution of IM5 (2.5 g, 17 mmol) in THF (20 mL) was added dropwise at −78° C. The reaction mixture was stirred for another 2 h at −78° C., then 2 h at room temperature. The solution was quenched with sat. NH₄Cl (50 mL), extracted with MTBE (50 mL×2). The combined organic layers were dried over Na₂SO₄, filtered and concentrated in vacuum. The residue was purified by silica gel chromatography (eluted: PE:EtOAc=3:1 to 1:1) to afford a crude product, and this was further purified by HPLC (method E modified: the gradient was 30-55% B for 0-10 min) to afford the intermediate (1.5 g, yield: 38%) as a yellow oil. ¹H NMR (CDCl₃): δ 7.23-7.26 (m, 2H), 6.84-6.88 (m, 2H), 4.58-4.62 (m, 1H), 4.06 (s, 1H), 3.79 (s, 3H), 3.44-3.53 (m, 2H), 3.38 (s, 3H), 1.20 (s, 9H). [α]_{D}²⁰=−120.0 (c=0.1, MeOH).

### Step 2:

To a solution of this intermediate (1.8 g, 6.3 mmol) in anhydrous dioxane (5 mL) at 0° C. was added HCl/dioxane (5 mL, 4.0 M) and the reaction was stirred at 0° C. for 1 h. The white precipitate was collected by filtration, washed with MTBE (20 mL×2), dried to afford IM8 (1.2 g, yield: 86%) as a white solid. ¹H NMR (DMSO-d₆): δ 8.56 (s-broad, 3H), 7.44-7.48 (m, 2H), 6.95-6.99 (m, 2H), 4.37-4.41 (m, 1H), 3.76 (s, 3H), 3.67-3.72 (m, 1H), 3.55-3.59 (m, 1H), 3.32 (s, 3H).

### IM9: 4-((R)-1-Amino-2-hydroxy-ethyl)-phenol hydrochloride

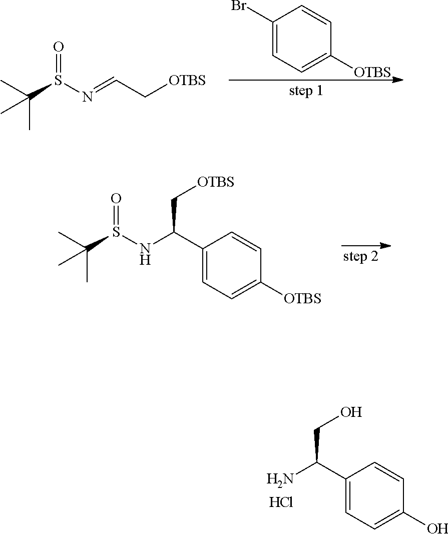 [SMILES: CC(C)(C)[S@@](=O)/N=C/CO[Si](C)(C)C(C)(C)C ; CC(C)(C)[Si](C)(C)Oc1ccc(Br)cc1 ; CC(C)(C)[S@@](=O)N[C@@H](CO[Si](C)(C)C(C)(C)C)c1ccc(O[Si](C)(C)C(C)(C)C)cc1 ; Cl ; N[C@@H](CO)c1ccc(O)cc1]

### Step 1:

To a solution of (4-Bromophenoxy)-tert-butyldimethylsilane (Sigma-Aldrich catalogue nr 444774, 10.0 g, 34.8 mmol) in anhydrous THF (100 mL) at −78° C. was added t-BuLi (53.6 mL, 69.6 mmol, 1.3 M in hexane) dropwise under N₂ protection over 1.5 h. The reaction mixture was stirred at −78° C. for 1 h. Then a solution of (R)-2-Methylpropane-2-sulfinic Acid [2-(tert-Butyldimethylsilanyloxy)ethylidene]amide (Tang, Tony. P et al., J. Org. Chem (2001) p 8772) (10.6 g, 38.3 mmol) in anhydrous THF (30 mL) was added thereto dropwise. The reaction mixture was stirred at −78° C. for another 4 h, and then quenched by addition of sat. NH₄Cl (80 mL). The mixture was extracted with EtOAc (100 mL×3). The combined organic phases were dried over anhydrous Na₂SO₄, filtered, concentrated under reduced pressure and purified via silica gel chromatography (eluting with PE:EtOAc=9:1 to 5:1) to afford the disilyl protected intermediate (10.12 g, yield: 60%) as a paint yellow oil. ¹H NMR (CDCl₃): δ7.23-7.29 (m, 2H), 6.84-6.88 (m, 2H), 4.51-4.54 (m, 1H), 4.42-4.47 (m, 1H), 3.79-3.86 (m, 1H), 3.65 (t, J=10.0 Hz, 1H), 1.28 (s, 9H), 1.04 (s, 9H), 0.79 (s, 9H), 0.25 (s, 6H), 0.12 (s, 6H).

### Step 2:

A solution of this intermediate (2.0 g, 4.12 mmol) in HCl/dioxane (20.0 mL, 4.0 M) was stirred at room temperature overnight. The solvent was removed under reduced pressure and the residue was washed with MTBE (20 mL×2), the precipitate was dried to afford crude IM9 (0.78 g) as grey solid.

### Example 1

### Preparation of Compounds of the Invention

### Compound 1: (1S,2S)-2-Phenyl-cyclopropanecarboxylic acid [(S)-1-(4-methoxy-phenyl)ethyl]-amide

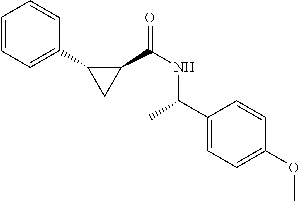 [SMILES: COc1ccc([C@H](C)NC(=O)[C@H]2C[C@@H]2c2ccccc2)cc1]

To a solution of IM1 (0.406 g, 2.50 mmol) in N,N-Dimethylformamide (8.00 mL, 103 mmol) was added (S)-1-(4-methoxy-phenyl)-ethylamine (0.344 g, 2.28 mmol) (Sigma-Aldrich, Catalog no 726656), N,N,N′,N′-Tetramethyl-O-(7-azabenzotriazol-1-yl)uronium Hexafluorophosphate (0.952 g, 2.50 mmol) and Triethylamine (0.476 mL, 3.41 mmol). The reaction was stirred at room temperature overnight. The reaction mixture was diluted with brine. Saturated NaHCO₃ solution was added until pH reached 8 and the mixture was extracted with EtOAc (3×50 mL). The combined organic layers were washed with brine (3×30 mL), dried over MgSO₄ and evaporated to dryness. Flash chromatography (silica, EtOAc/heptane 1:2) gave the title compound as a solid (0.22 g, 33%).

¹H NMR (500 MHz, DMSO) δ 8.48 (d, J=8.1 Hz, 1H), 7.31-7.20 (m, 4H), 7.18-7.13 (m, 1H), 7.10 (d, J=7.3 Hz, 2H), 6.87 (d, J=8.6 Hz, 2H), 4.97-4.83 (m, J=7.0 Hz, 1H), 3.72 (s, 3H), 2.27-2.14 (m, 1H), 1.97-1.90 (m, 1H), 1.40-1.30 (m, 4H), 1.23-1.10 (m, 1H). LCMS (m/z) 296.5 (MH⁺); t_{R}=0.71 min.

Compounds 2-5 were prepared analogously:

### Compound 2: (1S,2S)-2-Phenyl-cyclopropanecarboxylic acid ((S)-3-hydroxy-1-phenyl-propyl)-amide

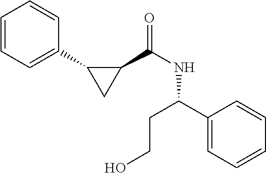 [SMILES: O=C(N[C@@H](CCO)c1ccccc1)[C@H]1C[C@@H]1c1ccccc1]

Prepared using IM1 and (S)-3-amino-3-phenyl-propan-1-ol (Ochem Inc., Catalog no 69A764). LCMS (m/z) 296.5 (MH⁺); t_{R}=0.62 min.

### Compound 3: (1S,2S)-2-Phenyl-cyclopropanecarboxylic acid [(S)-1-(4-fluoro-phenyl)-ethyl]-amide

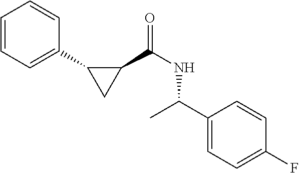 [SMILES: C[C@H](NC(=O)[C@H]1C[C@@H]1c1ccccc1)c1ccc(F)cc1]

Prepared using IM1 and (S)-1-(4-fluoro-phenyl)-ethylamine (Apollo Scientific, Catalog no PC0613).

LCMS (m/z) 284.5 (MH⁺); t_{R}=0.73 min.

### Compound 4: (1S,2S)-2-(3-Fluoro-phenyl)-cyclopropanecarboxylic acid [(R)-2-hydroxy-1-(4-methoxy-phenyl)-ethyl]-amide

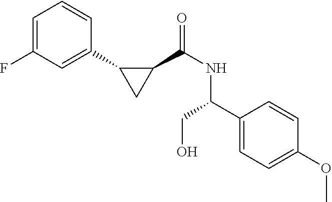 [SMILES: COc1ccc([C@H](CO)NC(=O)[C@H]2C[C@@H]2c2cccc(F)c2)cc1]

Prepared using IM3 and (R)-2-amino-2-(4-methoxy-phenyl)-ethanol (Asiba, Catalog no 10656).

LCMS (m/z) 330.5 (MH⁺); t_{R}=0.61 min.

### Compound 5: (1S,2S)-2-Phenyl-cyclopropanecarboxylic acid ((S)-1-p-tolyl-ethyl)-amide

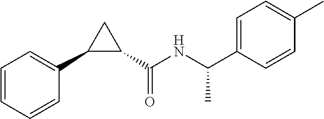 [SMILES: Cc1ccc([C@H](C)NC(=O)[C@H]2C[C@@H]2c2ccccc2)cc1]

Prepared using IM1 and (S)-1-p-tolyl-ethylamine (Sigma-Aldrich, Catalog no 726591). LCMS (m/z) 280.5 (MH⁺); t_{R}=0.77 min.

### Compound 6: (1S,2S)-2-Phenyl-cyclopropanecarboxylic acid [(S)-1-(3-fluoro-phenyl)-ethyl]-amide

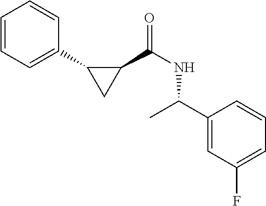 [SMILES: C[C@H](NC(=O)[C@H]1C[C@@H]1c1ccccc1)c1cccc(F)c1]

A mixture of (S)-1-(3-Fluoro-phenyl)-ethylamine (0.372 g, 2.67 mmol) (Apollo Scientific, catalog no PC3143), IM1 (0.650 g, 4.01 mmol) and N,N-Diisopropylethylamine (0.931 mL, 5.34 mmol) in Tetrahydrofuran (25.0 mL, 308 mmol) was degassed for 5 minutes with N₂. N-(3-Dimethylaminopropyl)-N′-ethylcarbodiimide hydrochloride (0.769 g, 4.01 mmol) and 1-Hydroxybenzotriazole (0.722 g, 5.34 mmol) were added as solids. The reaction mixture was stirred overnight at room temperature. The reaction mixture was diluted with water and extracted with EtOAc (3×80 mL). The combined organic layers were washed with brine (80 mL), dried over MgSO₄ and evaporated to dryness. Flash chromatography (silica, EtOAc/heptane 1:2) gave the title compound as a solid (0.18 g, 24%).

¹H NMR (500 MHz, DMSO) δ 8.59 (d, J=8.0 Hz, 1H), 7.40-7.32 (m, 1H), 7.31-7.24 (m, 2H), 7.22-7.11 (m, 5H), 7.08-6.99 (m, 1H), 5.02-4.90 (m, 1H), 2.25-2.17 (m, 1H), 1.98-1.91 (m, 1H), 1.37 (t, J=7.1 Hz, 4H), 1.24-1.18 (m, 1H). LCMS (m/z) 284.5 (MH+); tR=0.73 min.

Compounds 7-9 were prepared analogously:

### Compound 7: (1S,2S)-2-Phenyl-cyclopropanecarboxylic acid [(R)-2-hydroxy-1-(4-trifluoromethoxy-phenyl)-ethyl]-amide

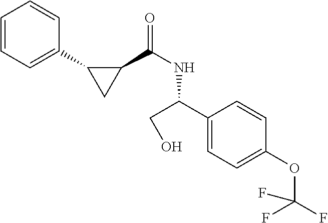 [SMILES: O=C(N[C@@H](CO)c1ccc(OC(F)(F)F)cc1)[C@H]1C[C@@H]1c1ccccc1]

Prepared using IM1 and (R)-2-amino-2-(4-trifluoromethoxy-phenyl)-ethanol (Netchem, Catalog no 514618).

LCMS (m/z) 366.5 (MH⁺); t_{R}=0.73 min.

### Compound 8: (1S,2S)-2-Phenyl-cyclopropanecarboxylic acid [(R)-1-(4-ethoxy-phenyl)-2-hydroxy-ethyl]-amide

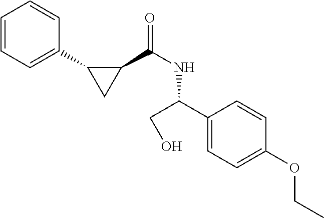 [SMILES: CCOc1ccc([C@H](CO)NC(=O)[C@H]2C[C@@H]2c2ccccc2)cc1]

Prepared using IM1 and (R)-2-amino-2-(4-ethoxy-phenyl)-ethanol (Netchem, Catalog no 514434).

LCMS (m/z) 326.5 (MH⁺); t_{R}=0.65 min.

### Compound 9: (1S,2S)-2-Phenyl-cyclopropanecarboxylic acid [(R)-1-(2-fluoro-4-methoxy-phenyl)-2-hydroxy-ethyl]-amide

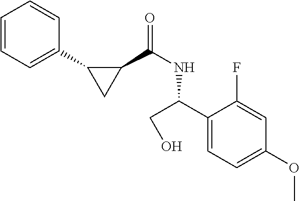 [SMILES: COc1ccc([C@H](CO)NC(=O)[C@H]2C[C@@H]2c2ccccc2)c(F)c1]

Prepared using IM1 and (R)-2-amino-2-(2-fluoro-4-methoxy-phenyl)-ethanol (Netchem, Catalog no 514788).

LCMS (m/z) 330.5 (MH⁺); t_{R}=0.62 min.

### Compound 10: (1S,2S)-2-Phenyl-cyclopropanecarboxylic acid [(R)-2-hydroxy-1-(4-methoxy-phenyl)-ethyl]-amide

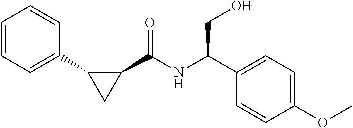 [SMILES: COc1ccc([C@H](CO)NC(=O)[C@H]2C[C@@H]2c2ccccc2)cc1]

To a mixture of compound (R)-2-Amino-2-(4-methoxy-phenyl)-ethanol (asiba, Catalog No Asiba, 10656) (2.15 g, 13.2 mmol) and HATU (5.47 g, 14.4 mmol) in DMF (20 mL) was added Et₃N (2.42 g, 24 mmol). The resulting mixture was kept at room temperature for 0.5 h. Compound IM1 (2.0 g, 12 mmol) was added and the resulting mixture was stirred room temperature for 5 h. The mixture was evaporated to dryness. Purification by preparative HPLC (Method E) to give the title compound (1.5 g, 47%). ¹H NMR (CDCl₃) δ 7.16-7.28 (m, 5H), 7.06-7.08 (m, 2H), 6.86-6.90 (m, 2H), 6.25 (d, 1H), 5.02-5.06 (m, 1H), 3.84-3.92 (m, 2H), 3.79 (s, 3H), 2.89 (d, 1H), 2.48-2.53 (m, 1H), 1.63-1.69 (m, 2H), 1.25-1.32 (m, 2H). [α]_{D}²⁰=+219.6° (c=0.1175, MeOH). LCMS (m/z) 312.2 (MH+); tR=0.60 min.

Compounds 11-15 were prepared analogously:

### Compound 11: (1S,2S)-2-Phenyl-cyclopropanecarboxylic acid [(S)-1-(3-methoxy-phenyl)ethyl]-amide

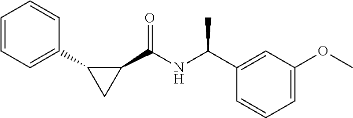 [SMILES: COc1cccc([C@H](C)NC(=O)[C@H]2C[C@@H]2c2ccccc2)c1]

Prepared using IM1 and (S)-1-(3-methoxy-phenyl)-ethylamine (Johnson Mattey, Catalog no 116324).

LCMS (m/z) 296.2 (MH⁺); t_{R}=0.73 min.

### Compound 12: (1S,2S)-2-Phenyl-cyclopropanecarboxylic acid [(S)-1-(2-fluoro-phenyl)-ethyl]-amide

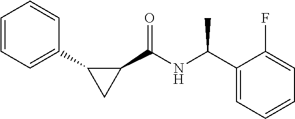 [SMILES: C[C@H](NC(=O)[C@H]1C[C@@H]1c1ccccc1)c1ccccc1F]

Prepared using IM1 and (S)-1-(2-fluoro-phenyl)-ethylamine (Apollo Scientific, Catalog no pc0612).

LCMS (m/z) 284.5 (MH⁺); t_{R}=0.73 min.

### Compound 13: (1S,2S)-2-(4-Fluoro-phenyl)-cyclopropanecarboxylic acid [(R)-2-hydroxy-1-(4-methoxy-phenyl)-ethyl]-amide

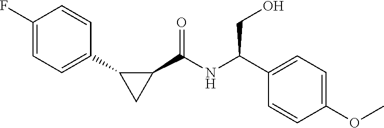 [SMILES: COc1ccc([C@H](CO)NC(=O)[C@H]2C[C@@H]2c2ccc(F)cc2)cc1]

Prepared using IM4 and (R)-2-amino-2-(4-methoxy-phenyl)-ethanol (Asiba, Catalog no 10656).

LCMS (m/z) 330.2 (MH⁺); t_{R}=0.62 min.

### Compound 14: (1S,2S)-2-(3-Fluoro-phenyl)-cyclopropanecarboxylic acid [(R)-2-hydroxy-1-(4-methoxy-phenyl)-ethyl]-amide

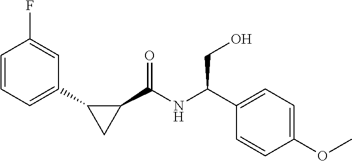 [SMILES: COc1ccc([C@H](CO)NC(=O)[C@H]2C[C@@H]2c2cccc(F)c2)cc1]

Prepared using IM3 and (R)-2-amino-2-(4-methoxy-phenyl)-ethanol (Asiba, Catalog no 10656).

LCMS (m/z) 330.2 (MH⁺); t_{R}=0.62 min.

### Compound 15: (1S,2S)-2-(2-Fluoro-phenyl)-cyclopropanecarboxylic acid [(R)-2-hydroxy-1-(4-methoxy-phenyl)-ethyl]-amide

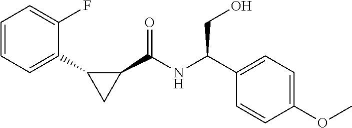 [SMILES: COc1ccc([C@H](CO)NC(=O)[C@H]2C[C@@H]2c2ccccc2F)cc1]

Prepared using IM2 and (R)-2-amino-2-(4-methoxy-phenyl)-ethanol (Asiba, Catalog no 10656).

LCMS (m/z) 330.2 (MH⁺); t_{R}=0.61 min.

### Compound 16: (1S,2S)-2-Phenyl-cyclopropanecarboxylic acid [(R)-2-methoxy-1-(4-methoxy-phenyl)-ethyl]-amide

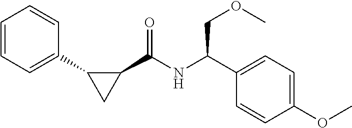 [SMILES: COC[C@H](NC(=O)[C@H]1C[C@@H]1c1ccccc1)c1ccc(OC)cc1]

To a solution of compound 10 (420 mg, 1.25 mmol) in anhydrous THF (4 mL) was added n-BuLi (0.6 mL, 1.5 mmol) dropwise at −78° C. over 20 min. Then Mel (191 mg, 1.34 mmol) was added and the mixture was stirred at room temperature for 6 h. An additional portion of Mel (191 mg, 1.34 mmol) was added and the mixture stirred 30 min. The reaction mixture was quenched with brine and extracted with methylene chloride (2×100 mL). The combined organic layers were dried over Na₂SO₄ and evaporated to dryness. Flash chromatography (silica, EtOAc:petroleum ether=1:2 to 2:1) gave the title compound as a colorless solid (180 mg, 41%). ¹H NMR (CDCl₃) δ 7.16-7.21 (m, 2H), 7.09-7.13 (m, 2H), 6.98-7.01 (m, 1H), 6.76-6.80 (m, 2H), 6.24 (d, 1H), 5.03-5.08 (m, 1H), 3.71 (s, 3H), 3.57 (d, 2H), 3.29 (s, 3H), 2.37-2.42 (m, 1H), 1.54-1.63 (m, 2H), 1.15-1.19 (m, 1H). [α]²⁰_{D}=+200.0° (c=0.1, MeOH). LCMS (m/z) 326.5 (MH⁺); t_{R}=0.69 min.

### Compound 17: (1S,2S)-2-Phenyl-cyclopropanecarboxylic acid [(R)-1-(4-ethoxy-phenyl)-2-methoxy-ethyl]-amide

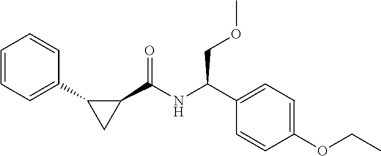 [SMILES: CCOc1ccc([C@H](COC)NC(=O)[C@H]2C[C@@H]2c2ccccc2)cc1]

To a solution of IM6 (230 mg, 1.0 mmol) in DMF (10 mL) with stirring was added Et₃N (303 mg, 3.0 mmol), IM1 (162 mg, 1.0 mmol), then HATU (420 mg, 1.1 mmol) at room temperature. The reaction was stirred at room temperature for 2 h. The mixture was separated by HPLC (Method F modified: Gradient was 37-67% B for 0-12 min with flow rate 25 ml/min) to afford compound 17 (240 mg, yield: 71%) as a white solid. ¹H NMR (CDCl₃): δ 7.14-7.30 (m, 5H), 7.05-7.10 (m, 2H), 6.81-6.88 (m, 2H), 6.32 (d, J=7.2 Hz, 1H), 5.09-5.14 (m, 1H), 4.00 (q, J=7.2 Hz, 2H), 3.63 (d, J=4.8 Hz, 2H), 3.36 (s, 3H), 2.43-2.48 (m, 1H), 1.60-1.69 (m, 2H), 1.39 (t, J=4.8 Hz, 3H), 1.21-1.27 (m, 1H). [α]_{D}²⁰=223.0 (c=0.1, MeOH). LCMS (m/z) 340.2 (MH⁺).

### Compound 18: (1S,2S)-2-Phenyl-cyclopropanecarboxylic acid [(R)-2-hydroxy-1-(4-hydroxy-phenyl)-ethyl]-amide

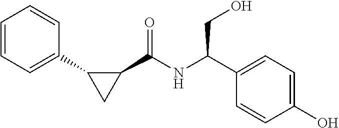 [SMILES: O=C(N[C@@H](CO)c1ccc(O)cc1)[C@H]1C[C@@H]1c1ccccc1]

To a solution of IM1 (300 mg, 1.85 mmol) and IM9 (525.3 mg, 2.77 mmol) in DMF (4 mL) was added Et₃N (935.8 mg, 9.25 mmol), then HATU (760 mg, 2.03 mmol) was added. The reaction mixture was stirred at room temperature overnight. The mixture was purified by HPLC (Method F modified: The column was an AD-5UM C18 150*30*5, and the gradient was 30-66% B for 0-10 min with flow rate 60 ml/min), and further by SFC (Method C modified: The column temp was 38° C., the pressure was 100 bar and the mobile phase was supercritical CO₂/MeOH+NH₂OH=80/20) to afford compound 18 (210 mg, yield: 38%) as a white solid. ¹H NMR (MeOH-d₄): δ 7.22-7.26 (m, 2H), 7.12-7.17 (m, 3H), 7.08-7.11 (m, 2H), 6.72-6.75 (m, 2H), 4.90-4.94 (m, 1H), 3.65-3.73 (m, 2H), 2.32-2.35 (m, 1H), 1.96-1.99 (m, 1H), 1.48-1.53 (m, 1H), 1.24-1.29 (m, 1H). [α]_{D}²⁰=179.0 (c=0.1, MeOH). LCMS (m/z) 298.1 (MH⁺).

### Compound 19: (1S,2S)-2-Phenyl-cyclopropanecarboxylic acid [(R)-1-(4-hydroxy-phenyl)-2-methoxy-ethyl]-amide

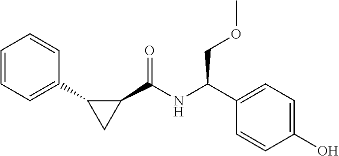 [SMILES: COC[C@H](NC(=O)[C@H]1C[C@@H]1c1ccccc1)c1ccc(O)cc1]

To a solution of IM7 (203 mg, 1.0 mmol) in DMF (5 mL) with stirring was added Et₃N (404 mg, 4.0 mmol), IM1 (162 mg, 1.0 mmol), then HATU (420 mg, 1.1 mmol) at room temperature. The reaction was stirred at room temperature for 2 h. The mixture was separated by HPLC (Method F modified: The gradient was 23-53% B for 0-12 min) to afford compound 19 (210 mg, yield: 67%) as a white solid. ¹H NMR (CDCl₃): δ 7.24-7.29 (m, 2H), 7.05-7.21 (m, 5H), 6.67-6.71 (m, 2H), 6.32 (d, J=7.2 Hz, 1H), 5.06-5.11 (m, 1H), 3.59-3.66 (m, 2H), 3.36 (s, 3H), 2.46-2.52 (m, 1H), 1.61-1.72 (m, 2H), 1.22-1.29 (m, 1H), [α]_{D}²⁰=200.0 (c=0.1, MeOH). LCMS (m/z) 312.1 (MH⁺).

### Compound 20: (1S,2S)-2-(4-Fluoro-phenyl)-cyclopropanecarboxylic acid [(R)-2-methoxy-1-(4-methoxy-phenyl)-ethyl]-amide

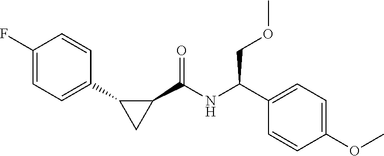 [SMILES: COC[C@H](NC(=O)[C@H]1C[C@@H]1c1ccc(F)cc1)c1ccc(OC)cc1]

To a solution of IM8 (240.00 mg, 1.33 mmol) and IM4 (290.00 mg, 1.33 mmol) in DMF (8 mL) was added Et₃N (0.95 mL, 6.66 mmol), then HATU (560 mg, 1.47 mmol). The reaction mixture was stirred at room temperature overnight. The crude product was purified by HPLC (method F modified: The column was an YMC-Actus Triart C18) and then further by SFC (Method C modified: The apparatus was a SFC-MA2, The column temp was 38° C., the pressure was 100 bar and the mobile phase was supercritical CO₂/MeOH+NH₂OH=85/15 with a flow of 50 ml/min) to afford compound 20 (384 mg, yield: 83.9%) as a white solid. ¹H NMR (CDCl₃): δ 7.24-7.28 (m, 2H), 6.99-7.04 (m, 2H), 6.92-6.97 (m, 2H), 6.84-6.88 (m, 2H), 6.33 (d, J=7.2 Hz, 1H), 5.10-5.15 (m, 1H), 3.78 (s, 3H), 3.64 (d, J=4.8 Hz, 2H), 3.36 (s, 3H), 2.42-2.49 (m, 1H), 1.61-1.64 (m, 2H), 1.17-1.22 (m, 1H). [α]_{D}²⁰=189.0 (c=0.1, MeOH). LCMS (m/z) 344.0 (MH⁺).

### Compound 21: (1S,2S)-2-(3-Fluoro-phenyl)-cyclopropanecarboxylic acid [(R)-2-methoxy-1-(4-methoxy-phenyl)-ethyl]-amide

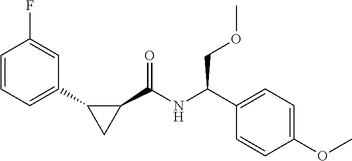 [SMILES: COC[C@H](NC(=O)[C@H]1C[C@@H]1c1cccc(F)c1)c1ccc(OC)cc1]

To a solution of IM8 (260 mg, 1.2 mmol) in DMF (5 mL) with stirring was added Et₃N (363 mg, 3.6 mmol), IM3 (216 mg, 1.2 mmol) and HATU (494 mg, 1.3 mmol) at room temperature. The reaction was stirred at room temperature for 2 hours. The mixture was separated by HPLC (Method F modified: The gradient was 30-55% B for 0-12 min and the flow was 80 ml/min) to afford Compound 21 (350 mg, yield: 85%) as a white solid. ¹H NMR (CDCl₃): δ 7.18-7.27 (m, 3H), 6.84-6.89 (m, 4H), 6.71-6.75 (m, 1H), 6.32 (d, J=7.2 Hz, 1H), 5.09-5.14 (m, 1H), 3.78 (s, 3H), 3.62-3.65 (m, 2H), 3.37 (s, 3H), 2.43-2.48 (m, 1H), 1.61-1.69 (m, 2H), 1.19-1.25 (m, 1H). [α]_{D}²⁰=192.0 (c=0.1, MeOH). LCMS (m/z) 344.1 (MH⁺).

### Compound 22: (1S,2S)-2-(4-Fluoro-phenyl)-cyclopropanecarboxylic acid [(R)-2-hydroxy-1-(4-hydroxy-phenyl)-ethyl]-amide

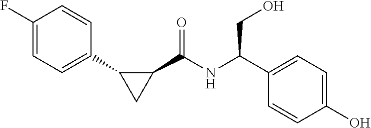 [SMILES: O=C(N[C@@H](CO)c1ccc(O)cc1)[C@H]1C[C@@H]1c1ccc(F)cc1]

To a solution of IM9 (240 mg, 1.27 mmol) and IM4 (190 mg, 1.05 mmol) in DMF (5 mL) was added Et₃N (561.6 mg, 5.55 mmol), then HATU (441 mg, 1.16 mmol). The reaction was stirred at room temperature for 4 hours. The crude product was purified by HPLC (Method F modified: the column was an AD-5UM C18, and the gradient was 38-69% B for 0-10 min) and further by SFC (Method C modified: The pressure was 100 bar and the mobile phase was supercritical CO₂/MeOH+NH₂OH=80/20) to afford Compound 22 (192 mg, yield: 58%) as a white solid. ¹H NMR (MeOH-d₄): δ 7.09-7.17 (m, 4H), 6.94-7.00 (m, 2H), 7.08-7.11 (m, 2H), 6.72-6.76 (m, 1H), 4.90-4.94 (m, 1H), 3.65-3.73 (m, 2H), 2.30-2.36 (m, 1H), 1.92-1.97 (m, 1H), 1.47-1.52 (m, 1H), 1.19-1.25 (m, 1H) [α]_{D}²⁰=189.0 (c=0.1, MeOH). LCMS (m/z) 316.1 (MH⁺).

### Compound 23: (1S,2S)-2-(3-Fluoro-phenyl)-cyclopropanecarboxylic acid [(R)-2-hydroxy-1-(4-hydroxy-phenyl)-ethyl]-amide

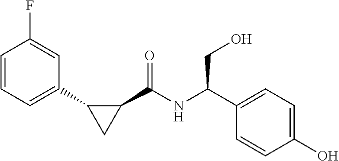 [SMILES: O=C(N[C@@H](CO)c1ccc(O)cc1)[C@H]1C[C@@H]1c1cccc(F)c1]

To a solution of IM9 (400.54 mg, 2.11 mmol) and IM3 (274.46 mg, 1.52 mmol) in DMF (6 mL) was added Et₃N (770.7 mg, 7.62 mmol) and HATU (441 mg, 1.16 mmol). The reaction mixture was stirred at room temperature for 4 hours. The mixture was purified by HPLC (Method F modified: the column was an AD-5UM C18, and the gradient was 45-68% B for 0-10 min, flow rate 60 ml/min) and further by SFC (Method C modified: The column was a Chiracel OJ 250×30 mm, and the temp was 38° C., the pressure was 100 bar and the mobile phase was supercritical CO₂/MeOH+NH₂OH=70/30) to afford Compound 23 (236.2 mg, yield: 49%) as a white solid. ¹H NMR (MeOH-d₄): δ 7.22-7.28 (m, 1H), 7.13-7.18 (m, 2H), 6.81-6.95 (m, 3H), 6.72-6.76 (m, 2H), 4.92 (t, J=6.4 Hz, 1H), 3.64-3.73 (m, 2H), 2.31-2.36 (m, 1H), 1.98-2.03 (m, 1H), 1.50-1.55 (m, 1H), 1.24-1.28 (m, 1H). [α]_{D}²⁰=138.0 (c=0.1, MeOH). LC-MS (m/z) 316.1 (MH⁺).

### Compound 24: (1S,2S)-2-Phenyl-cyclopropanecarboxylic acid {(R)-1-[4-(2-fluoro-ethoxy)phenyl]-2-hydroxy-ethyl}-amide

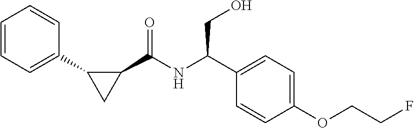 [SMILES: O=C(N[C@@H](CO)c1ccc(OCCF)cc1)[C@H]1C[C@@H]1c1ccccc1]

To a solution of compound 18 (160.00 mg, 0.54 mmol) and Cs₂CO₃ (350.60 mg, 1.08 mmol) in CH₃CN (16 mL) was added 1-Fluoro-2-iodo-ethane (140.00 mg, 0.81 mmol). The reaction was stirred at 80° C. for 3 h. The mixture was filtered and the filtrate was purified by HPLC (method F modified: The column was an YMC-Actus Triart C18, with a gradient of 35-65% B for 0-10 min and the flow rate was 25 ml/min) to afford Compound 24 (141.0 mg, yield: 76.2%) as a white solid. ¹H NMR (MeOH-d₄): δ 7.18-7.23 (m, 4H), 7.09-7.13 (m, 1H), 7.04-7.07 (m, 2H), 6.86-6.89 (m, 2H), 4.92 (t, J=6.8 Hz, 1H), 4.70-4.72 (m, 1H), 4.58-4.60 (m, 1H), 4.16-4.19 (m, 1H), 4.09-4.12 (m, 1H), 3.65-3.69 (m, 2H), 2.27-2.29 (m, 1H), 1.93-1.98 (m, 1H), 1.44-1.49 (m, 1H), 1.19-1.24 (m, 1H). [α]_{D}²⁰=176.0 (c=0.1, MeOH). LC-MS (m/z) 344.1 (MH⁺).

### Compound 25: (1S,2S)-2-(4-Fluoro-phenyl)-cyclopropanecarboxylic acid {(R)-1-[4-(2-fluoro-ethoxy)-phenyl]-2-hydroxy-ethyl}-amide

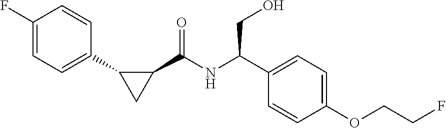 [SMILES: O=C(N[C@@H](CO)c1ccc(OCCF)cc1)[C@H]1C[C@@H]1c1ccc(F)cc1]

To a solution of compound 22 (160.00 mg, 0.51 mmol) and Cs₂CO₃ (350.60 mg, 1.08 mmol) in CH₃CN (16 mL) was added 1-fluoro-2-iodoethane (132.40 mg, 0.76 mmol). The reaction mixture was stirred at 80° C. for 3 h. The mixture was filtered and the filtrate was purified via silica gel chromatography (eluent EtOAc) to afford compound compound 25 (147 mg, yield: 80%) as a white solid. ¹H NMR (CDCl₃): δ 7.22-7.26 (m, 2H), 7.00-7.05 (m, 2H), 6.90-7.00 (m, 4H), 6.23 (d, J=7.2 Hz, 1H) 5.02-5.07 (m, 1H), 4.79-4.82 (m, 1H), 4.67-4.70 (m, 1H), 4.22-4.25 (m, 1H), 4.15-4.18 (m, 1H), 3.85-3.93 (m, 2H), 2.47-2.52 (m, 1H), 1.59-1.66 (m, 2H), 1.21-1.28 (m, 1H). [α]_{D}²⁰=154.0 (c=0.1, MeOH). LC-MS (m/z) 362.2 (MH⁺).

### In Vitro Assays

The nicotinic acetylcholine receptor α7 is a calcium-permeable ion channel, whose activity can be measured by over expression in mammalian cells or oocytes. These two individual assays are described in Example 2 and 3, respectively.

### Example 2

### α7 NNR Flux Assay

In this version of the assay, the human α7 receptor is stably expressed in the rat GH4C1 cell line. The assay was used to identify positive allosteric modulators (PAMs) of the α7 receptor. Activation of the channel was measured by loading cells with the calcium-sensitive fluorescent dye Calcium-4 (Assay kit from Molecular Devices), and then measuring real-time changes in fluorescence upon treatment with test compounds.

The cell line ChanClone GH4C1-nAChRalphα7 from Genionics was seeded from frozen stock in 384-well plates in culture media 2-3 days before experiment to form an approximately 80% confluent layer on the day of experiment.

### Cell Plating and Dye Loading

The cell culture was split into “22.5 cm×22.5 cm”-plates with approximately 100×10³ cells/cm². After four days incubation in a humidified incubator at 37° C. and 5% CO₂, it had grown to an 80-90% confluent layer, and the cells were harvested.

Culture Media:

500 mL DMEM/F12 (Gibco 31331)

50 mL FBS (Gibco 10091-155, lot 453269FD)

5 mL Sodium Pyruvate (Gibco 11360)

5 mL Pen/Strep (Gibco 15140)

0.1 mg/mL G-418 (Gibco 11811-064)

Two or three days before the experiment the cells were seeded in 384 well plates from Greiner bio-one (781946, CELLCOAT, Poly-D-Lysine, black, μClear).

The media was poured off and the plate washed with PBS and left to drain. 5 mL Trypsin was added, cells were washed and incubated (at room temperature) for about 10 seconds. Trypsin was poured of quickly and the cells were incubated for 2 minutes at 37° C. (if the cells were not already detached). Cells were resuspended in 10 mL culture media and transferred to 50 mL tubes.

The cell suspension was counted (NucleoCounter, total cell count) from the first plates to estimate the total cell number of the whole batch.

The cells were seeded in 384 well plates with 30 μL/well (30000 cells/well) while stirring the cell suspension or otherwise preventing the cells from precipitating.

The plates were incubated at room temperature for 30-45 minutes.

The plates were placed in incubator for two days (37° C. and 5% CO₂).

### Loading the Cells

The loading buffer was 5% v/v Calcium-4 Kit and 2.5 mM Probenecid in assay buffer.

190 mL assay buffer

10 mL Kit-solution

2 mL 250 mM Probenecid

This volume was enough for 3×8 cell plates.

Culture media were removed from the cell plates and 20 μL loading buffer was added in each well. The cell plates were placed in trays and incubated 90 minutes in the incubator (37° C.). Thereafter the plates were incubated 30 minutes at room temperature, still protected from light.

Now the cell plates were ready to run in the Functional Drug Screening System (FDSS).

The assay buffer was HBSS with 20 mM HEPES, pH 7.4 and 3 mM CaCl₂.

### FDSS Ca Assay

200 nL 10 mM compound solution in DMSO was diluted in 50 μL assay buffer. The final test concentrations in the cell plates were 20-10-5-2.5-1.25-0.625-0.312-0.156-0.078-0.039 μM. Assay buffer and 3 μM PNU-120596 were used for control.

The agonist acetylcholine was added to a final concentration of 20 μM (˜E0100). In the FDSS7000 the Ex480-Em540 was measured with 1 second intervals. The baseline was made of 5 frames before addition of test compounds, and 95 frames more were made before addition of acetylcholine. The measurement stopped 30 frames after the 2^{nd} addition.
Raw data for each well were collected as “the maximum fluorescence count” in the interval 100-131 seconds and as “the average fluorescence count” in the interval 96-100 seconds. The positive allosteric modulation in the 2^{nd} addition was the enhancement of agonist response with test compound compared to agonist alone.

Results were calculated as % modulation of test compound compared to the reference PNU-120596 set to 100%. From these data EC₅₀ curves were generated giving EC₅₀, hill and maximum stimulation.

The compounds of the invention were shown to be PAMs of the α7 receptor. The compounds of the present invention characterized in the flux assay generally possess EC₅₀ values below 20.000 nM or less such as below 10.000 nM. Many compounds, in fact have EC₅₀ values below 5.000 nM. Table 1 shows EC₅₀ values for exemplified compounds of the invention.

TABLE 1

| Compound | EC₅₀ (nM) |
|---|---|
| 1 | 1500 |
| 2 | 4900 |
| 3 | 2400 |
| 4 | 1200 |
| 5 | 3800 |
| 6 | 3100 |
| 7 | 3600 |
| 8 | 270 |
| 9 | 610 |
| 10 | 645 |
| 11 | 3000 |
| 12 | 1900 |
| 13 | 510 |
| 14 | 400 |
| 15 | 1200 |
| 16 | 160 |
| 17 | 33 |
| 18 | 3900 |
| 19 | 3600 |
| 20 | 84 |
| 21 | 1100 |
| 22 | 3200 |
| 23 | 3800 |
| 24 | 1500 |
| 25 | 140 |

### Example 3

### α7NNR Oocyte Assay

Expression of α7 nACh Receptors in Xenopus Oocytes.

Oocytes are surgically removed from mature female Xenopus laevis anaesthetized in 0.4% MS-222 for 10-15 min. The oocytes are then digested at room temperature for 2-3 hours with 0.5 mg/mL collagenase (type IA Sigma-Aldrich) in OR2 buffer (82.5 mM NaCl, 2.0 mM KCl, 1.0 mM MgCl₂ and 5.0 mM HEPES, pH 7.6). Oocytes avoid of the follicle layer are-selected and incubated for 24 hours in Modified Barth's Saline buffer (88 mM NaCl, 1 mM KCl, 15 mM HEPES, 2.4 mM NaHCO₃, 0.41 mM CaCl₂, 0.82 mM MgSO₄, 0.3 mM Ca(NO₃)₂) supplemented with 2 mM sodium pyruvate, 0.1 U/I penicillin and 0.1 μg/l streptomycin. Stage IV oocytes are identified and injected with 4.2-48 nl of nuclease free water containing 0.1-1.2 ng of cRNA coding for human α7 nACh receptors or 3.0-32 ng of cRNA coding for rat α7 nACh receptors and incubated at 18° C. for 1-10 days when they are used for electrophysiological recordings.

Electrophysiological Recordings of α7 nACh Receptors Expressed in Oocytes.

Oocytes are used for electrophysiological recordings 1-10 days after injection. Oocytes are placed in a 1 mL bath and perfused with Ringer buffer (115 mM NaCl, 2.5 mM KCl, 10 mM HEPES, 1.8 mM CaCl₂, 0.1 mM MgCl₂, pH 7.5). Cells are impaled with agar plugged 0.2-1 MO electrodes containing 3 M KCl and voltage clamped at −90 mV by a GeneClamp 500B amplifier. The experiments are performed at room temperature. Oocytes are continuously perfused with Ringer buffer and the drugs are applied in the perfusate. ACh (30 μM) applied for 30 sec are used as the standard agonist for activation of the α7 nACh receptors. In the standard screening set-up the new test compound (10 μM or 30 μM) are applied for 1 min of pre-application allowing for evaluation of agonistic activity followed by 30 sec of co-application with ACh (30 μM) allowing for evaluation of PAM activity. The response of co-application was compared to the agonistic response obtained with ACh alone. The drug induced effects on both the peak response and the total charge (AUC) response arecalculated thus giving the effect of drug induced PAM activity as fold modulation of the control response.

For more elaborate studies doses-response curves can be performed for evaluation of max-fold modulation and EC₅₀ values for both peak and AUC responses.

1. A compound according to formula [I] 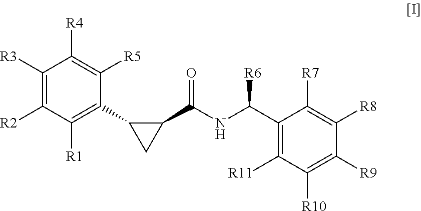
wherein R1, R2, R3, R4 and R5 are selected independently of each other from H and fluorine;
R6 is selected from methyl, methoxymethyl, hydroxymethyl and hydroxyethyl;
R7, R8, R9, R10 and R11 are selected independently of each other from H, C₁₋₆alkyl, C₂₋₆alkenyl, C₂₋₆alkynyl, C₁₋₆alkoxy, hydroxy, cyano, NR12R13, halogen and OR14, wherein said C₁₋₆alkyl, C₂₋₆alkenyl, C₂₋₆alkynyl or C₁₋₆alkoxy is optionally substituted with one or more substituents selected from chlorine, fluorine, C₁₋₆alkoxy, cyano and NR12R13;
R12 and R13 independently represent hydrogen, C₁₋₆alkyl, C₂₋₆alkenyl and C₂₋₆alkynyl;
R14 represents a monocyclic saturated ring moiety having 4-6 ring atoms wherein one of said ring atoms is O and the other ring atoms are C;
and pharmaceutically acceptable salts thereof;
with the proviso that the compound of formula [I] is other than
(1S,2S)-2-Phenyl-cyclopropanecarboxylic acid{(R)-1-[4-(2-ethyl-butoxy)-2-methoxy-phenyl]-2-hydroxy-ethyl}-amide;
(1S,2S)-2-Phenyl-cyclopropanecarboxylic acid ((R)-2-hydroxy-1-phenyl-ethyl)-amide;
(1S,2S)-2-Phenyl-cyclopropanecarboxylic acid ((S)-1-phenyl-ethyl)-amide.

2. The compound according to claim 1, wherein four or more of R1, R2, R3, R4 and R5 are H.

3. The compound according to claim 1, wherein one of R1, R2, R3, R4 and R5 is represented by fluorine and the remaining of R1, R2, R3, R4 and R5 are represented by H.

4. The compound according to claim 1, wherein R7, R8, R9, R10 and R11 are selected independently of each other from H, C₁₋₆alkoxy, hydroxy and fluorine, wherein said C₁₋₆alkoxy is optionally substituted with one or more fluorine.

5. The compound according to claim 1, wherein R7, R8, R9, R10 and R11 are selected independently of each other from H, methoxy, ethoxy, trifluoromethoxy, 2-fluoroethoxy, hydroxy and fluorine.

6. The compound according to claim 1, wherein three or more of R7, R8, R9, R10 and R11 are H.

7. The compound according to claim 1, wherein R6 is methyl.

8. The compound according to claim 1, wherein R6 is methoxymethyl.

9. The compound according to claim 1, wherein R6 is hydroxymethyl.

10. The compound according to claim 1, wherein R6 is hydroxyethyl.

11. The compound according to claim 1 wherein the compound is selected from the group consisting of
1: (1S,2S)-2-Phenyl-cyclopropanecarboxylic acid [(S)-1-(4-methoxy-phenyl)-ethyl]-amide;
2: (1S,2S)-2-Phenyl-cyclopropanecarboxylic acid ((S)-3-hydroxy-1-phenyl-propyl)amide;
3: (1S,2S)-2-Phenyl-cyclopropanecarboxylic acid [(S)-1-(4-fluoro-phenyl)-ethyl]-amide;
4: (1S,2S)-2-(3-Fluoro-phenyl)-cyclopropanecarboxylic acid [(R)-2-hydroxy-1-(4-methoxy-phenyl)-ethyl]-amide;
5: (1S,2S)-2-Phenyl-cyclopropanecarboxylic acid ((S)-1-p-tolyl-ethyl)-amide;
6: (1S,2S)-2-Phenyl-cyclopropanecarboxylic acid [(S)-1-(3-fluoro-phenyl)-ethyl]-amide;
7: (1S,2S)-2-Phenyl-cyclopropanecarboxylic acid [(R)-2-hydroxy-1-(4-trifluoromethoxy-phenyl)-ethyl]-amide;
8: (1S,2S)-2-Phenyl-cyclopropanecarboxylic acid [(R)-1-(4-ethoxy-phenyl)-2-hydroxy-ethyl]-amide;
9: (1S,2S)-2-Phenyl-cyclopropanecarboxylic acid [(R)-1-(2-fluoro-4-methoxy-phenyl)-2-hydroxy-ethyl]-amide;
10: (1S,2S)-2-Phenyl-cyclopropanecarboxylic acid [(R)-2-hydroxy-1-(4-methoxy-phenyl)-ethyl]-amide;
11: (1S,2S)-2-Phenyl-cyclopropanecarboxylic acid [(S)-1-(3-methoxy-phenyl)-ethyl]-amide;
12: (1S,2S)-2-Phenyl-cyclopropanecarboxylic acid [(S)-1-(2-fluoro-phenyl)-ethyl]-amide;
13: (1S,2S)-2-(4-Fluoro-phenyl)-cyclopropanecarboxylic acid [(R)-2-hydroxy-1-(4-methoxy-phenyl)-ethyl]-amide;
14: (1S,2S)-2-(3-Fluoro-phenyl)-cyclopropanecarboxylic acid [(R)-2-hydroxy-1-(4-methoxy-phenyl)-ethyl]-amide;
15: (1S,2S)-2-(2-Fluoro-phenyl)-cyclopropanecarboxylic acid [(R)-2-hydroxy-1-(4-methoxy-phenyl)-ethyl]-amide;
16: (1S,2S)-2-Phenyl-cyclopropanecarboxylic acid [(R)-2-methoxy-1-(4-methoxy-phenyl)-ethyl]-amide;
17: (1S,2S)-2-Phenyl-cyclopropanecarboxylic acid [(R)-1-(4-ethoxy-phenyl)-2-methoxy-ethyl]-amide;
18: (1S,2S)-2-Phenyl-cyclopropanecarboxylic acid [(R)-2-hydroxy-1-(4-hydroxy-phenyl)ethyl]-amide;
19: (1S,2S)-2-Phenyl-cyclopropanecarboxylic acid [(R)-1-(4-hydroxy-phenyl)-2-methoxy-ethyl]-amide;
20: (1S,2S)-2-(4-Fluoro-phenyl)-cyclopropanecarboxylic acid [(R)-2-methoxy-1-(4-methoxy-phenyl)-ethyl]-amide;
21: (1S,2S)-2-(3-Fluoro-phenyl)-cyclopropanecarboxylic acid [(R)-2-methoxy-1-(4-methoxy-phenyl)-ethyl]-amide;
22: (1S,2S)-2-(4-Fluoro-phenyl)-cyclopropanecarboxylic acid [(R)-2-hydroxy-1-(4-hydroxy-phenyl)-ethyl]-amide;
23: (1S,2S)-2-(3-Fluoro-phenyl)-cyclopropanecarboxylic acid [(R)-2-hydroxy-1-(4-hydroxy-phenyl)-ethyl]-amide;
24: (1S,2S)-2-Phenyl-cyclopropanecarboxylic acid {(R)-1-[4-(2-fluoro-ethoxy)-phenyl]-2-hydroxy-ethyl}-amide; and
25: (1S,2S)-2-(4-Fluoro-phenyl)-cyclopropanecarboxylic acid {(R)-1-[4-(2-fluoro-ethoxy)phenyl]-2-hydroxy-ethyl}-amide;
or a pharmaceutically acceptable salt thereof.

12. (canceled)

13. A method of treating a disease or disorder comprising administering an effective amount of a compound of claim 1 to a patient in need thereof, wherein the disease or disorder is selected from the group consisting of psychosis; schizophrenia; cognitive disorders; cognitive impairment associated with schizophrenia; attention deficit hyperactivity disorder (ADHD); autism spectrum disorders, Alzheimer's disease (AD); mild cognitive impairment (MCI); age associated memory impairment (AAMI); senile dementia; AIDS dementia; Pick's disease; dementia associated with Lewy bodies; dementia associated with Down's syndrome; Huntington's disease; Parkinson's disease (PD); obsessive-compulsive disorder (OCD); traumatic brain injury; epilepsy; post-traumatic stress; Wernicke-Korsakoff syndrome (WKS); post-traumatic amnesia; cognitive deficits associated with depression; diabetes, weight control, inflammatory disorders, reduced angiogenesis; amyotrophic lateral sclerosis and pain.

14. A pharmaceutical composition comprising a compound according to claim 1, and one or more pharmaceutically acceptable carrier or excipient.

15. (canceled)

16. The compound of claim 1 wherein the compound is
(1S,2S)-2-Phenyl-cyclopropanecarboxylic acid [(R)-2-hydroxy-1-(4-methoxy-phenyl)ethyl]-amide or a pharmaceutically acceptable salt thereof.

17. The method of claim 13 wherein the disease or disorder is schizophrenia.

18. The method of claim 13 wherein the disease or disorder is a cognitive disorder.

19. The method of claim 13 wherein the disease or disorder is cognitive impairment associated with schizophrenia.

20. The method of claim 13 wherein the disease or disorder is mild cognitive impairment (MCI).

21. The method of claim 13 wherein the disease or disorder is pain.
